SEQUENCE LISTING (53 sequences)

SEQ ID NO: 1 - DNA, 29 bp, synthetic construct
  source 1..29: mol_type = other DNA
aatgatacggcgaccaccgagatctacac

SEQ ID NO: 2 - DNA, 24 bp, synthetic construct
  source 1..24: mol_type = other DNA
caagcagaagacggcatacgagat

SEQ ID NO: 3 - DNA, 29 bp, synthetic construct
  source 1..29: mol_type = other DNA
gtgtagatctcggtggtcgccgtatcatt

SEQ ID NO: 4 - DNA, 24 bp, synthetic construct
  source 1..24: mol_type = other DNA
atctcgtatgccgtcttctgcttg

SEQ ID NO: 5 - DNA, 17 bp, synthetic construct
  source 1..17: mol_type = other DNA
aatgatacggcgaccga

SEQ ID NO: 6 - DNA, 17 bp, synthetic construct
  source 1..17: mol_type = other DNA
tcggtcgccgtatcatt

SEQ ID NO: 7 - DNA, 14 bp, synthetic construct
  source 1..14: mol_type = other DNA
tcgtcggcagcgtc

SEQ ID NO: 8 - DNA, 15 bp, synthetic construct
  source 1..15: mol_type = other DNA
gtctcgtgggctcgg

SEQ ID NO: 9 - DNA, 33 bp, synthetic construct
  source 1..33: mol_type = other DNA
tcgtcggcagcgtcagatgtgtataagagacag

SEQ ID NO: 10 - DNA, 34 bp, synthetic construct
  source 1..34: mol_type = other DNA
gtctcgtgggctcggagatgtgtataagagacag

SEQ ID NO: 11 - DNA, 24 bp, synthetic construct
  source 1..24: mol_type = other DNA
gagtaagtggaagagataggaagg

SEQ ID NO: 12 - DNA, 43 bp, synthetic construct
  source 1..43: mol_type = other DNA
gagtaagtggaagagataggaaggagatgtgtataagagacag

SEQ ID NO: 13 - DNA, 24 bp, synthetic construct
  source 1..24: mol_type = other DNA
ccttcctatctcttccacttactc

SEQ ID NO: 14 - DNA, 43 bp, synthetic construct
  source 1..43: mol_type = other DNA
ccttcctatctcttccacttactcagatgtgtataagagacag

SEQ ID NO: 15 - DNA, 23 bp, synthetic construct
  source 1..23: mol_type = other DNA
  misc_binding 23: bound_moiety = 3' propanol
ccttcctatctcttccacttact

SEQ ID NO: 16 - DNA, 43 bp, synthetic construct; identical to SEQ ID NO: 14
  source 1..43: mol_type = other DNA
  misc_binding 43: bound_moiety = 3' propanol

SEQ ID NO: 17 - DNA, 33 bp, synthetic construct
  source 1..33: mol_type = other DNA
ctgtctcttatacacatctgacgctgccgacga

SEQ ID NO: 18 - DNA, 14 bp, synthetic construct
  source 1..14: mol_type = other DNA
gacgctgccgacga

SEQ ID NO: 19 - DNA, 34 bp, synthetic construct
  source 1..34: mol_type = other DNA
ctgtctcttatacacatctccgagcccacgagac

SEQ ID NO: 20 - DNA, 15 bp, synthetic construct
  source 1..15: mol_type = other DNA
ccgagcccacgagac

SEQ ID NO: 21 - DNA, 43 bp, synthetic construct
  source 1..43: mol_type = other DNA
ctgtctcttatacacatctgagtaagtggaagagataggaagg

SEQ ID NO: 22 - DNA, 43 bp, synthetic construct
  source 1..43: mol_type = other DNA
ctgtctcttatacacatctccttcctatctcttccacttactc

SEQ ID NO: 23 - DNA, 37 bp, synthetic construct
  source 1..37: mol_type = other DNA
  modified_base 31: mod_base = OTHER; note = 2-deoxyuridine
ttttttttttaatgatacggcgaccaccganctacac

SEQ ID NO: 24 - DNA, 34 bp, synthetic construct
  source 1..34: mol_type = other DNA
  modified_base 32: mod_base = OTHER; note = 8-oxoguanine
ttttttttttcaagcagaagacggcatacgagat

SEQ ID NO: 25 - DNA, 30 bp, synthetic construct
  source 1..30: mol_type = other DNA
agtgtagatctcggtggtcgccgtatcatt

SEQ ID NO: 26 - DNA, 30 bp, synthetic construct
  source 1..30: mol_type = other DNA
tgtgtagatctcggtggtcgccgtatcatt

SEQ ID NO: 27 - DNA, 30 bp, synthetic construct
  source 1..30: mol_type = other DNA
cgtgtagatctcggtggtcgccgtatcatt

SEQ ID NO: 28 - DNA, 30 bp, synthetic construct
  source 1..30: mol_type = other DNA
ggtgtagatctcggtggtcgccgtatcatt

SEQ ID NO: 29 - DNA, 25 bp, synthetic construct
  source 1..25: mol_type = other DNA
aatctcgtatgccgtcttctgcttg

SEQ ID NO: 30 - DNA, 25 bp, synthetic construct
  source 1..25: mol_type = other DNA
tatctcgtatgccgtcttctgcttg

SEQ ID NO: 31 - DNA, 25 bp, synthetic construct
  source 1..25: mol_type = other DNA
catctcgtatgccgtcttctgcttg

SEQ ID NO: 32 - DNA, 25 bp, synthetic construct
  source 1..25: mol_type = other DNA
gatctcgtatgccgtcttctgcttg

SEQ ID NO: 33 - DNA, 18 bp, synthetic construct
  source 1..18: mol_type = other DNA
atcggtcgccgtatcatt

SEQ ID NO: 34 - DNA, 18 bp, synthetic construct
  source 1..18: mol_type = other DNA
ttcggtcgccgtatcatt

SEQ ID NO: 35 - DNA, 18 bp, synthetic construct
  source 1..18: mol_type = other DNA
ctcggtcgccgtatcatt

SEQ ID NO: 36 - DNA, 18 bp, synthetic construct
  source 1..18: mol_type = other DNA
gtcggtcgccgtatcatt

SEQ ID NO: 37 - DNA, 33 bp, synthetic construct
  source 1..33: mol_type = other DNA
acactctttccctacacgacgctcttccgatct

SEQ ID NO: 38 - DNA, 33 bp, synthetic construct
  source 1..33: mol_type = other DNA
agatcggaagagcgtcgtgtagggaaagagtgt

SEQ ID NO: 39 - DNA, 34 bp, synthetic construct
  source 1..34: mol_type = other DNA
gtgactggagttcagacgtgtgctcttccgatct

SEQ ID NO: 40 - DNA, 34 bp, synthetic construct
  source 1..34: mol_type = other DNA
agatcggaagagcacacgtctgaactccagtcac

SEQ ID NO: 41 - DNA, 35 bp, synthetic construct
  source 1..35: mol_type = other DNA
  misc_binding 1: bound_moiety = 5' dual-biotin
ttttttaatgatacggcgaccaccgagatctacac

SEQ ID NO: 42 - DNA, 30 bp, synthetic construct
  source 1..30: mol_type = other DNA
  misc_binding 1: bound_moiety = 5' dual-biotin
ttttttcaagcagaagacggcatacgagat

SEQ ID NO: 43 - DNA, 30 bp, synthetic construct
  source 1..30: mol_type = other DNA
  misc_difference 29^30: note = phosphorothioate linkage
aatgatacggcgaccaccgagatctacact

SEQ ID NO: 44 - DNA, 24 bp, synthetic construct
  source 1..24: mol_type = other DNA
  misc_difference 23^24: note = phosphorothioate linkage
caagcagaagacggcatacgagat

SEQ ID NO: 45 - DNA, 151 bp, Homo sapiens
  source 1..151: mol_type = genomic DNA
cccaggggaggttcccaggaaggcataggctaaaggtcaacaagaacagtgcaaaccccagcgggtgaccaaggagagggatcatttgcattaaggcacaaagtcaacaggagtggctgcagagcttacagagcaggacacaggacccaga

SEQ ID NO: 46 - DNA, 151 bp, Homo sapiens
  source 1..151: mol_type = genomic DNA
cccaggggaggttcccaggaaggcataggctaaaggtcaacaagaacagtgcaaaccccagcgggtgaccaaggagagggatcatttgcattaaggcacaaagtcaacaggagtggctgcagagctcacagagcaggacacaggacccaga

SEQ ID NO: 47 - DNA, 149 bp, Escherichia virus phiX174
  source 1..149: mol_type = genomic DNA
ccgtggacagatttgtcattgtgagcattttcatcccgaagttgcggctcattctgattctgaacagcttcttgggaagtagcgacagcttggtttttagtgagttgttccattctttagctcccagacctttagcagcaaggtccata

SEQ ID NO: 48 - DNA, 149 bp, Escherichia virus phiX174
  source 1..149: mol_type = genomic DNA
ccgtggacagatttgtcattgtgagcattttcatcccgaagttgcggctcattctgattctgaacagcttcttgggaagtagcgacagcttggtttttagtgagttgttccattctttagctcctagacctttagcagcaaggtccata

SEQ ID NO: 49 - DNA, 151 bp, Escherichia virus phiX174
  source 1..151: mol_type = genomic DNA
atacgttaacaaaaagtcagatatggaccttgctgctaaaggtctaggagctaaagaatggaacaactcactaaaaaccaagctgtcgctacttcccaagaagctgttcagaatcagaatgagccgcaacttcgggatgaaaatgctcaca

SEQ ID NO: 50 - DNA, 151 bp, Escherichia virus phiX174
  source 1..151: mol_type = genomic DNA
aacccagcttggtaagttggattaagcactccgtggacagatttgtcattgtgagcattttcatcccaagttgcggctcattctgattctgaacagcttcttgggaagtagccgacagcttggtttttagtgagttgttccattctttagc

SEQ ID NO: 51 - DNA, 150 bp, Escherichia virus phiX174
  source 1..150: mol_type = genomic DNA
aggattgacaccctcccaattgtatgttttcatgcctccaaatcttggaggcttttttatggttcgttcttattacccttctgaatgtcacgctgattattttgactttgagcgtatcgaggctcttaaacctgctattgaggcttgtgg

SEQ ID NO: 52 - DNA, 150 bp, Escherichia virus phiX174
  source 1..150: mol_type = genomic DNA
tagtgtaattaatatcaagttgggggagcacattgtagcattgtgccaattcatccattaacttctcagtaacagattcaaactcatcacgaacgtcagaagcaggcttatggccgtcaacatacatatcaccattatcgtactcaacga

SEQ ID NO: 53 - DNA, 150 bp, Escherichia virus phiX174
  source 1..150: mol_type = genomic DNA
tagtgttattaatatcaagttgggggagcacattgtagcattgtgccaattcatccattaacttctcagtaacagatacaaactcatcacgaacgtcagaagcaggcttatggccgtcaacatacatatcaccattatcgaactcaacga

(19) United States
(12) Patent Application Publication
(10) Pub. No.: US 2024/0360503 A1
(43) Pub. Date: Oct. 31, 2024

(54) METHODS FOR PREPARING SIGNALS FOR CONCURRENT SEQUENCING

(71) Applicant: Illumina, Inc., San Diego, CA (US)

(72) Inventors: Gery VESSERE, San Diego, CA (US); Aathavan KARUNAKARAN, San Diego, CA (US); Come RACZY, San Diego, CA (US); Jeff GAU, San Diego, CA (US); Jonathan BOUTELL, Cambridge (GB); Roberto ANDRES, Cambridge (GB); Michael BUREK, San Diego, CA (US); Eli CARRAMI, Cambridge (GB)

(21) Appl. No.: 18/573,770

(22) PCT Filed: Mar. 15, 2023
(86) PCT No.: PCT/EP2023/056626

§ 371 Date: Dec. 22, 2023

Related U.S. Application Data
(60) Provisional application No. 63/439,417, filed on Jan. 17, 2023.
(60) Provisional application No. 63/439,438, filed on Jan. 17, 2023.
(60) Provisional application No. 63/439,443, filed on Jan. 17, 2023.
(60) Provisional application No. 63/439,466, filed on Jan. 17, 2023.
(60) Provisional application No. 63/439,501, filed on Jan. 17, 2023.
(60) Provisional application No. 63/439,519, filed on Jan. 17, 2023.
(60) Provisional application No. 63/439,522, filed on Jan. 17, 2023.
(60) Provisional application No. 63/439,491, filed on Jan. 17, 2023.
(60) Provisional application No. 63/269,383, filed on Mar. 15, 2022.
(60) Provisional application No. 63/439,415, filed on Jan. 17, 2023.

(51) Int. Cl. C12Q 1/6874; C12Q 1/6806; G16B 40/10

(52) U.S. Cl. CPC C12Q 1/6874; C12Q 1/6806; G16B 40/10

(57) ABSTRACT

The invention relates to methods for use in nucleic acid sequencing, in particular methods for use in concurrent sequencing.

### FIELD OF THE INVENTION

The invention relates to methods for use in nucleic acid sequencing, in particular methods for use in concurrent sequencing.

### BACKGROUND OF THE INVENTION

In some types of next-generation sequencing (NGS) technologies, a nucleic acid cluster is created on a flow cell by amplifying an original template nucleic acid strand. Sequencing cycles may be performed as complementary strands of the template nucleic acids are being synthesized, i.e., using sequencing-by-synthesis (SBS) processes.

In each sequencing cycle, deoxyribonucleic acid analogs conjugated to fluorescent labels are hybridised to the template nucleic acids, and excitation light sources are used to excite the fluorescent labels on the deoxyribonucleic acid analogs. Detectors capture fluorescent emissions from the fluorescent labels and identify the deoxyribonucleic acid analogs. As a result, the sequence of the template nucleic acids may be determined by repeatedly performing such sequencing cycles.

NGS allows for the sequencing of a number of different template nucleic acids simultaneously, which has significantly reduced the cost of sequencing in the last twenty years. However, there remains a desire for further improvements in sequencing throughput and speed.

### SUMMARY OF THE INVENTION

According to an aspect of the present invention, there is provided a method of preparing at least one polynucleotide sequence for identification, comprising:
selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

In one embodiment, the concentration of the first portions capable of generating the first signal is greater than a concentration of the second portions capable of generating the second signal.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 base pairs and the second portion is at least 25 base pairs.

In one embodiment, the ratio between the concentration of the first portions capable of generating the first signal and the concentration of the second portions capable of generating the second signal is between 1.25:1 to 5:1, between 1.5:1 to 3:1, or about 2:1.

In one embodiment, the method comprises selectively processing at least one polynucleotide sequence comprising a first portion and a second portion.

In one embodiment, the method comprises selectively processing at least one first polynucleotide sequence comprising a first portion and at least one second polynucleotide sequence comprising a second portion.

In one embodiment, the first signal and the second signal are spatially unresolved.

In one embodiment, the at least one polynucleotide sequence comprises portions of a double-stranded nucleic acid template, and each of the first portions comprise a forward strand of the template, and each of the second portions comprise a reverse strand of the template or a forward complement strand of the template; or wherein each of the first portions comprise a reverse strand of the template, and each of the second portions comprise a forward strand of the template or a reverse complement strand of the template.

In one embodiment, the at least one polynucleotide sequence comprises portions of a double-stranded nucleic acid template, and each of the first portions comprises a forward strand of a template, and each of the second portions comprises a reverse complement strand of the template; or wherein each of the first portions comprises a reverse strand of a template, and each of the second portions comprises a forward complement strand of the template.

In one embodiment, the at least one polynucleotide sequence comprising the first portion and the second portion, or the at least one first polynucleotide sequence comprising the first portion and the at least one second polynucleotide sequence comprising the second portion, is/are attached to a solid support. In one embodiment, the solid support is a flow cell.

In one embodiment the at least one polynucleotide sequence comprising the first portion and the second portion, or the at least one first polynucleotide sequence comprising the first portion and the at least one second polynucleotide sequence comprising the second portion, form a cluster on the solid support.

In one embodiment, the cluster is formed by bridge amplification.

In one embodiment, the at least one polynucleotide sequence comprising the first portion and the second portion forms a monoclonal cluster.

In one embodiment, the at least one first polynucleotide sequence comprising the first portion and the at least one second polynucleotide sequence comprising the second portion form a duoclonal cluster.

In one embodiment, a first region occupied by the at least one first polynucleotide sequence comprising the first portion within the duoclonal cluster is the same as, or substantially overlapping with, a second region occupied by the at least one second polynucleotide sequence comprising the second portion within the duoclonal cluster.

In one embodiment, the solid support comprises at least one first immobilised primer and at least one second immobilised primer.

In one embodiment, the first immobilised primer comprises a sequence as defined in SEQ ID NO: 1 or 5, or a variant or fragment thereof; and the second immobilised primer comprises a sequence as defined in SEQ ID NO: 2, or a variant or fragment thereof.

In one embodiment, the method comprises selectively processing at least one polynucleotide sequence comprising a first portion and a second portion, and wherein each polynucleotide sequence is attached to a first immobilised primer.

In one embodiment, each polynucleotide sequence comprising the first portion and the second portion further comprises a second adaptor sequence, wherein the second adaptor sequence is substantially complementary to the second immobilised primer.

In one embodiment, the method comprises selectively processing at least one first polynucleotide sequence comprising a first portion and at least one second polynucleotide sequence comprising a second portion, wherein each first polynucleotide sequence is attached to a first immobilised primer, and wherein each second polynucleotide sequence is attached a second immobilised primer.

In one embodiment each first polynucleotide sequence comprises a second adaptor sequence and each second polynucleotide sequence comprises a first adaptor sequence, wherein the second adaptor sequence is substantially complementary to the second immobilised primer and wherein the first adaptor sequence is substantially complementary to the first immobilised primer.

In one embodiment, selectively processing comprises conducting selective sequencing. In another embodiment, selectively processing comprises conducting selective amplification.

In one embodiment, selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

In one embodiment, the blocked second primer comprises a blocking group at a 3′ end of the blocked second primer. In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In one embodiment, the blocked second primer comprises a sequence as defined in SEQ ID NO: 11 to 16 or a variant or fragment thereof and/or the unblocked second primer comprises a sequence as defined in SEQ ID NO: 11 to 14 or a variant or fragment thereof.

In one embodiment, the blocked second primer comprises a sequence as defined in SEQ ID NO: 13 or 14 or a variant or fragment thereof and/or the unblocked second primer comprises a sequence as defined in SEQ ID NO: 15 or 16 or a variant or fragment thereof.

In one embodiment, selective processing comprises selectively removing some or substantially all of second immobilised primers that are not yet extended, and conducting a further amplification cycle in order to selectively amplify the first polynucleotide sequence(s) relative to the second polynucleotide sequence(s).

In one embodiment, selectively processing comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

In one embodiment, the primer blocking agent is added whilst first polynucleotide sequence(s) are hybridised to the second immobilised primers.

In one embodiment, the method comprises contacting some or substantially all of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to the 5′ additional nucleotide.

In one embodiment, the primer blocking agent is a blocked nucleotide. In one embodiment, the blocked nucleotide comprises a blocking group at a 3′ end of the blocked nucleotide.

In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase. In one embodiment, the blocked nucleotide is A or G.

In another aspect of the invention, there is provided a method of sequencing at least one polynucleotide sequence, comprising:
preparing at least one polynucleotide sequence for identification using a method as described herein; and
concurrently sequencing nucleobases in the first portion and the second portion based on the intensity of the first signal and the intensity of the second signal.

In one embodiment, concurrently sequencing nucleobases comprises performing sequencing-by-synthesis or sequencing-by-ligation.

In one embodiment, the method further comprises a step of conducting paired-end reads.

In one embodiment, the step of concurrently sequencing nucleobases comprises:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In embodiments, selecting the classification based on the first and second intensity data comprises selecting the classification based on the combined intensity of the first and second signal components and the combined intensity of the third and fourth signal components.

In embodiments, the plurality of classifications comprises sixteen classifications, each classification representing one of sixteen unique combinations of first and second nucleobases.

In embodiments, the first signal component, second signal component, third signal component and fourth signal component are generated based on light emissions associated with the respective nucleobase.

In one embodiment, the light emissions are detected by a sensor, wherein the sensor is configured to provide a single output based upon the first and second signals.

In one embodiment, the sensor comprises a single sensing element.

In embodiments, the method further comprises repeating steps (a) to (d) for each of a plurality of base calling cycles.

In another aspect of the invention, there is provided a primer, wherein the primer comprises a sequence as defined in SEQ ID NO: 11 to 16, or a variant or fragment thereof.

In one embodiment the 3′ end of the primer comprises a 3′-OH group. In one embodiment, the 3′ end of the primer comprises a blocking group at a 3′ end of the primer. In one embodiment, the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

In another aspect of the invention, there is provided use of a primer as described herein in preparing at least one polynucleotide sequence for identification as described herein.

In another aspect of the invention, there is provided a kit comprising instructions for preparing at least one polynucleotide sequence for identification as described herein; and/or sequencing at least one polynucleotide sequence as described herein.

In one embodiment the kit further comprises a primer as described herein.

In one embodiment the kit further comprises an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

In one embodiment the kit further comprises a primer-blocking agent(s), wherein the primer-blocking agent is a blocked nucleotide, or a blocked A or G.

In one embodiment the kit further comprises at least one extended primer sequence(s), wherein the extended primer sequence is selected from SEQ ID NO: 25 to 36, and wherein the extended primer sequence further comprise a 5′ additional nucleotide, wherein the 5′ additional nucleotide is complementary to the primer-blocking agent.

In another aspect of the invention, there is provided an amplification composition comprising a recombinase, a DNA polymerase, a single-stranded DNA binding protein (SSB) and primer-blocking agent, wherein the primer-blocking agent, in one aspect, is a blocked nucleotide, or, in another aspect, a blocked A or G.

In one embodiment, the amplification composition further comprises at least one extended primer sequence(s), wherein the extended primer sequence is selected from SEQ ID NO: 25 to 36, and wherein the extended primer sequence further comprises a 5′ additional nucleotide, wherein the 5′ additional nucleotide is complementary to the primer-blocking agent.

In another aspect of the invention, there is provided a data processing device comprising means for carrying out a method as described herein.

In one embodiment, the data processing device is a polynucleotide sequencer.

In another aspect of the invention, there is provided a computer program product comprising instructions, which when the program is executed by a processor, cause the processor to carry out a method as described herein.

In another aspect of the invention, there is provided a computer-readable storage medium comprising instructions which, when executed by a processor, cause the processor to carry out a method as described herein.

In another aspect of the invention, there is provided a computer-readable data carrier having stored thereon a computer program product as described herein.

In another aspect of the invention, there is provided a data carrier signal carrying a computer program product as described herein.

### DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 shows a forward strand, reverse strand, forward complement strand, and reverse complement strand of a polynucleotide molecule.

FIG. 2 shows an example of a polynucleotide sequence (or insert) with 5′ and 3′ adaptor sequences.

FIG. 3 shows a typical polynucleotide with 5′ and 3′ adaptor sequences.

FIG. 4 shows an example of PCR stitching. Here, two sequences—a strand of a human library and a strand of a phiX library are joined together to create a single polynucleotide strand comprising both a first portion (comprising the strand of the human sequence) and a second portion (comprising the strand of the phiX sequence), as well as terminal and internal adaptor sequences.

FIG. 5 shows the preparation of a concatenated polynucleotide sequence comprising a first portion and a second portion using a tandem insert method, comprising (A) preparation of a desired first (forked) adaptor and second (forked) adaptor from three oligos; (B) different types of first (forked) adaptors and second (forked) adaptors that do not anneal to each other due to the presence of a third oligo on at least one of the first (forked) adaptor and/or the second (forked) adaptor; (C) ligation of the template polynucleotide strand and adaptors generates three products, with the desired product containing both types of adaptor being produced at a proportion of 50%; (D) synthesis of concatenated strands from the desired product; and (E) completion of the synthesis of the concatenated strands from the desired product.

FIG. 6 shows the preparation of a concatenated polynucleotide sequence comprising a first portion and a second portion using a loop fork method.

FIG. 7 shows an example of a concatenated polynucleotide sequence comprising a first portion and a second portion, as well as terminal and internal adaptor sequences.

FIG. 8 shows an example of a concatenated polynucleotide sequence comprising a first portion and a second portion, as well as terminal and internal adaptor sequences.

FIG. 9 shows a typical solid support.

FIG. 10 shows the stages of bridge amplification and the generation of an amplified cluster comprising (A) a library strand hybridising to an immobilised primer; (B) generation of a template strand from the library strand; (C) dehybridisation and washing away the library strand; (D) hybridisation of the template strand to another immobilised primer; (E) generation of a template complement strand from the template strand via bridge amplification; (F) dehybridisation of the sequence bridge; (G) hybridisation of the template strand and template complement strand to immobilised primers; and (H) subsequent bridge amplification to provide a plurality of template and template complement strands.

FIG. 11 shows the stages of bridge amplification for concatenated polynucleotide sequences and the generation of an amplified cluster, comprising (A) a concatenated library strand hybridising to a immobilised primer; (B) generation of a template strand from the library strand; (C) dehybridisation and washing away the library strand; (D) generation of a template complement strand from the template strand via bridge amplification and dehybridisation of the sequence bridge; (E) further amplification to provide a plurality of template and template complement strands; and (F) cleavage of one set of the template and template complement strands.

FIG. 12 shows the detection of nucleobases using 4-channel, 2-channel and 1-channel chemistry.

FIG. 13 shows a method of selective sequencing.

FIG. 14 shows a method of selective amplification comprising (A) selective cleavage of one type of immobilised primer from the support; (B) only template (or template complement) strands complementary to the free immobilised primer anneal and undergo bridge amplification, (C) producing different proportions of template and template complement strands; (D) subsequent standard (non-selective) sequencing occurs in different proportions enabling signal differentiation.

FIG. 15 shows a method of selective amplification comprising (A) template and template complement strands annealing to immobilised primers; (B) addition of a primer-blocking agent that binds only to one type of immobilised primer, preventing the extension from that one type of immobilised primer, preventing the extension from one type of immobilised primer; (C) producing different proportions of template and template complement strands; (D) subsequent standard (non-selective) sequencing occurs in different proportions enabling signal differentiation.

FIG. 16 shows a method of selective amplification comprising (A) flowing a (or a plurality of) extended primer sequence(s) containing at least one additional 5′ nucleotide across the surface of the solid support; (B) addition of a primer-blocking agent that binds only to one type of immobilised primer and is complementary to the additional 5′ nucleotide of the extended primer sequence, preventing the extension from one type of immobilised primer.

FIG. 17 shows (A) that by plotting relative intensities of light signals obtained from a first channel (ch1) and a second channel (ch2), a constellation of 16 clouds is obtained; (B) alignment of R1 and R2 (minor and major reads respectively) with the known human and PhiX sequence.

FIG. 18 shows that by plotting relative intensities of light signals obtained from a first channel (ch1) and a second channel (ch2), a constellation of 16 clouds is obtained.

FIG. 19 shows (A) that by plotting relative intensities of light signals obtained from a first channel (ch1) and a second channel (ch2), a constellation of 16 clouds is obtained for R1 and R2 concurrently and R3 and R4 concurrently; (B) alignment of R1, R2, R3 and R4 with the known sequence; (C) annotation of where R1, R2, R3 and R4 appear on the known sequence.

FIG. 20 is a plot showing graphical representations of sixteen distributions of signals generated by polynucleotide sequences according to one embodiment.

FIG. 21 is a flow diagram showing a method for base calling according to one embodiment.

### DETAILED DESCRIPTION OF THE INVENTION

All patents, patent applications, and other publications referred to herein, including all sequences disclosed within these references, are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

The present invention can be used in sequencing, in particular concurrent sequencing. Methodologies applicable to the present invention have been described in WO 08/041002, WO 07/052006, WO 98/44151, WO 00/18957, WO 02/06456, WO 07/107710, WO 05/068656, U.S. Ser. No. 13/661,524 and US 2012/0316086, the contents of which are herein incorporated by reference. Further information can be found in US 20060024681, US20060292611, WO 06/110855, WO 06/135342, WO 03/074734, WO 07/010252, WO 07/091077, WO 00/179553, WO 98/44152 and WO 2022/087150, the contents of which are herein incorporated by reference.

As used herein, the term “variant” refers to a variant polypeptide sequence or part of the polypeptide sequence that retains desired function of the full non-variant sequence. For example, a desired function of the immobilised primer retains the ability to bind (i.e. hybridise) to a target sequence.

As used in any aspect described herein, a “variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid sequence. The sequence identity of a variant can be determined using any number of sequence alignment programs known in the art. As an example, Emboss Stretcher from the EMBL-EBI may be used: https://www.ebi.ac.uk/Tools/psa/emboss_stretcher/ (using default parameters: pair output format, Matrix=BLOSUM62, Gap open=1, Gap extend=1 for proteins; pair output format, Matrix=DNAfull, Gap open=16, Gap extend=4 for nucleotides).

As used herein, the term “fragment” refers to a functionally active series of consecutive nucleic acids from a longer nucleic acid sequence. The fragment may be at least 99%, at least 95%, at least 90%, at least 80%, at least 70%, at least 60%, at least 50%, at least 40% or at least 30% the length of the longer nucleic acid sequence. In one embodiment, a fragment as used herein also retains the ability to bind (i.e. hybridise) to a target sequence.

Sequencing generally comprises four fundamental steps: 1) library preparation to form a plurality of target polynucleotides for identification; 2) cluster generation to form an array of amplified template polynucleotides; 3) sequencing the cluster array of amplified template polynucleotides; and 4) data analysis to identify characteristics of the target polynucleotides from the amplified template polynucleotide sequences. These steps are described in greater detail below.

### Library Strands and Template Terminology

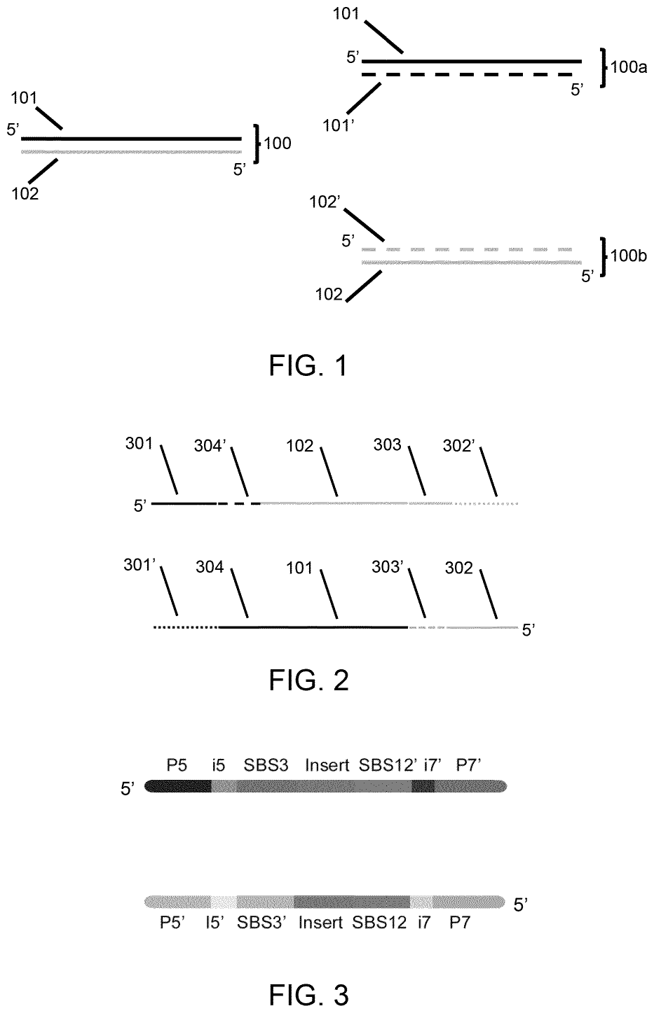

For a given double-stranded polynucleotide sequence 100 to be identified, the polynucleotide sequence 100 comprises a forward strand of the sequence 101 and a reverse strand of the sequence 102. See FIG. 1.

When the polynucleotide sequence 100 is replicated (e.g. using a DNA/RNA polymerase), complementary versions of the forward strand 101 of the sequence 100 and the reverse strand 102 of the sequence 100 are generated. Thus, replication of the polynucleotide sequence 100 provides a double-stranded polynucleotide sequence 100a that comprises a forward strand of the sequence 101 and a forward complement strand of the sequence 101′, and a double-stranded polynucleotide sequence 100b that comprises a reverse strand of the sequence 102 and a reverse complement strand of the sequence 102′.

The term “template” may be used to describe a complementary version of the double-stranded polynucleotide sequence 100. As such, the “template” comprises a forward complement strand of the sequence 101′ and a reverse complement strand of the sequence 102′. Thus, by using the forward complement strand of the sequence 101′ as a template for complementary base pairing, a sequencing process (e.g. a sequencing-by-synthesis or a sequencing-by-ligation process) reproduces information that was present in the original forward strand of the sequence 101. Similarly, by using the reverse complement strand of the sequence 102′ as a template for complementary base pairing, a sequencing process (e.g. a sequencing-by-synthesis or a sequencing-by-ligation process) reproduces information that was present in the original reverse strand of the sequence 102.

The two strands in the template may also be referred to as a forward strand of the template 101′ and a reverse strand of the template 102′. The complement of the forward strand of the template 101′ is termed the forward complement strand of the template 101, whilst the complement of the reverse strand of the template 102′ is termed the reverse complement strand of the template 102.

Generally, where forward strand, reverse strand, forward complement strand, and reverse complement strand are used herein without qualifying whether they are with respect to the original polynucleotide sequence 100 or with respect to the “template”, these terms may be interpreted as referring to the “template”.

| Language for original | Corresponding language for the |
|---|---|
| polynucleotide sequence 100 | “template” |
| Forward strand of the sequence | Forward complement strand of the |
| 101 | template 101 (sometimes referred to |
| | herein as forward complement |
| | strand 101) |
| Reverse strand of the sequence | Reverse complement strand of the |
| 102 | template 102 (sometimes referred to |
| | herein as reverse complement |
| | strand 102) |
| Forward complement strand of | Forward strand of the template 101′ |
| the sequence 101′ | (sometimes referred to herein as |
| | forward strand 101′) |
| Reverse complement strand of | Reverse strand of the template 102′ |
| the sequence 102′ | (sometimes referred to herein as |
| | reverse strand 102′) |

### Library Preparation

Library preparation is the first step in any high-throughput sequencing platform. These libraries allow templates to be generated via complementary base pairing that can subsequently be clustered and amplified. During library preparation, nucleic acid sequences, for example genomic DNA sample, or cDNA or RNA sample, is converted into a sequencing library, which can then be sequenced. By way of example with a DNA sample, the first step in library preparation is random fragmentation of the DNA sample. Sample DNA is first fragmented and the fragments of a specific size (typically 200-500 bp, but can be larger) are ligated, sub-cloned or “inserted” in-between two oligo adaptors (adaptor sequences). The original sample DNA fragments are referred to as “inserts”. The target polynucleotides may advantageously also be size-fractionated prior to modification with the adaptor sequences.

As described herein, typically the templates to be generated from the libraries may include a concatenated polynucleotide sequence comprising a first portion and a second portion. Alternatively, the templates to be generated typically include separate polynucleotide sequences, in particular a first polynucleotide sequence comprising a first portion and a second polynucleotide sequence comprising a second portion. Generating these templates from particular libraries may be performed according to methods known to persons of skill in the art. However, some example approaches of preparing libraries suitable for generation of such templates are described below.

In some embodiments, the library may be prepared by ligating adaptor sequences to double-stranded polynucleotide sequences, each comprising a forward strand of the sequence and a reverse strand of the sequence, as described in more detail in e.g. WO 07/052006, which is incorporated herein by reference. In some cases, “tagmentation” can be used to attach the sample DNA to the adaptors, as described in more detail in e.g. WO 10/048605, US 2012/0301925, US 2013/0143774 and WO 2016/189331, each of which are incorporated herein by reference. In tagmentation, double-stranded DNA is simultaneously fragmented and tagged with adaptor sequences and PCR primer binding sites. The combined reaction eliminates the need for a separate mechanical shearing step during library preparation. These procedures may be used, for example, for preparing templates including a first polynucleotide sequence comprising a first portion and a second polynucleotide sequence comprising a second portion, wherein the first portion is a forward strand of the template, and the second portion is a forward complement strand of the template—i.e. a copy of the forward strand (or alternatively, wherein the first portion is a reverse strand of the template, and the second portion is a reverse complement strand of the template).

Where features herein are described in relation to the “forward” strand, it should be considered that these features could equally be applied to the “reverse strand”.

Where libraries are prepared by ligating adaptor sequences to double-stranded polynucleotide sequences as described above, library preparation may comprise ligating a first primer-binding sequence 301′ (e.g. P5′, such as SEQ ID NO: 3) and a second terminal sequencing primer binding site 304 (e.g. SBS3′, for example, SEQ ID NO: 38) to a 3′-end of a forward strand of a sequence 101. See FIG. 2. The library preparation may be arranged such that the second terminal sequencing primer binding site 304 is attached (e.g. directly attached) to the 3′-end of the forward strand of the sequence 101, and such that the first primer-binding sequence 301′ is attached (e.g. directly attached) to the 3′-end of the second terminal sequencing primer binding site 304.

The library preparation may further comprise ligating a complement of first terminal sequencing primer binding site 303′ (e.g. SBS12, such as SEQ ID NO: 39) (also referred to herein as a first terminal sequencing primer binding site complement 303′) and a complement of a second primer-binding sequence 302 (also referred to herein as a second primer-binding complement sequence 302) (e.g. P7, such as SEQ ID NO: 2) to a 5′-end of the forward strand of the sequence 101. The library preparation may be arranged such that first terminal sequencing primer binding site complement 303′ is attached (e.g. directly attached) to the 5′-end of the forward strand of the sequence 101, and such that second primer-binding complement sequence 302 is attached (e.g. directly attached) to the 5′-end of first terminal sequencing primer binding site complement 303′.

Thus, one strand of a polynucleotide within a polynucleotide library may comprise, in a 5′ to 3′ direction, a second primer-binding complement sequence 302 (e.g. P7), a first terminal sequencing primer binding site complement 303′ (e.g. SBS12), a forward strand of the sequence 101, a second terminal sequencing primer binding site 304 (e.g. SBS3′), and a first primer-binding sequence 301′ (e.g. P5′) (FIG. 2—bottom strand).

Although not shown in FIG. 2, the strand may further comprise one or more index sequences. As such, a first index sequence (e.g. i7) may be provided between the second primer-binding complement sequence 302 (e.g. P7) and the first terminal sequencing primer binding site complement 303′ (e.g. SBS12). Separately, or in addition, a second index complement sequence (e.g. i5′) may be provided between the second terminal sequencing primer binding site 304 (e.g. SBS3′) and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), a first terminal sequencing primer binding site complement 303′ (e.g. SBS12), a forward strand of the sequence 101, a second terminal sequencing primer binding site 304 (e.g. SBS3′), a second index complement sequence (e.g. i5′), and a first primer-binding sequence 301′ (e.g. P5′). A typical polynucleotide is shown in FIG. 3 (bottom strand).

When a double-stranded sequence 100 is used, the library preparation may also comprise ligating a second primer-binding sequence 302′ (e.g. P7′) and a first terminal sequencing primer binding site 303 (e.g. SBS12′) to a 3′-end of a reverse strand of a sequence 102. The library preparation may be arranged such that first terminal sequencing primer binding site 303 is attached (e.g. directly attached) to the 3′-end of the reverse strand of the sequence 102, and such that the second primer-binding sequence 302′ is attached (e.g. directly attached) to the 3′-end of first terminal sequencing primer binding site 303.

The library preparation may further comprise ligating a complement of a second terminal sequencing primer binding site 304′ (e.g. SBS3) (also referred to herein as a second terminal sequencing primer binding site complement 304′) and a complement of a first primer-binding sequence 301 (also referred to herein as a first primer-binding complement sequence 301) (e.g. P5) to a 5′-end of the reverse strand of the sequence 102. The library preparation may be arranged such that the second terminal sequencing primer binding site complement 304′ is attached (e.g. directly attached) to the 5′-end of the reverse strand of the sequence 102, and such that the first primer-binding complement sequence 301 is attached (e.g. directly attached) to the 5′-end of the second terminal sequencing primer binding site complement 304′.

Thus, another strand of a polynucleotide within a polynucleotide library may comprise, in a 5′ to 3′ direction, a first primer-binding complement sequence 301 (e.g. P5), a second terminal sequencing primer binding site complement 304′ (e.g. SBS3), a reverse strand of the sequence 102, a first terminal sequencing primer binding site 303 (e.g. SBS12′), and a second primer-binding sequence 302′ (e.g. P7′) (FIG. 2—top strand).

Although not shown in FIG. 2, the another strand may further comprise one or more index sequences. As such, a second index sequence (e.g. i5) may be provided between the first primer-binding complement sequence 301 (e.g. P5) and the second terminal sequencing primer binding site complement 304′ (e.g. SBS3). Separately, or in addition, a first index complement sequence (e.g. i7′) may be provided between the first terminal sequencing primer binding site 303 (e.g. SBS12′) and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), a second terminal sequencing primer binding site complement 304′ (e.g. SBS3), a reverse strand of the sequence 102, a first terminal sequencing primer binding site 303 (e.g. SBS12′), a first index complement sequence (e.g. i7′), and a second primer-binding sequence 302′ (e.g. P7′). A typical polynucleotide is shown in FIG. 3 (top strand).

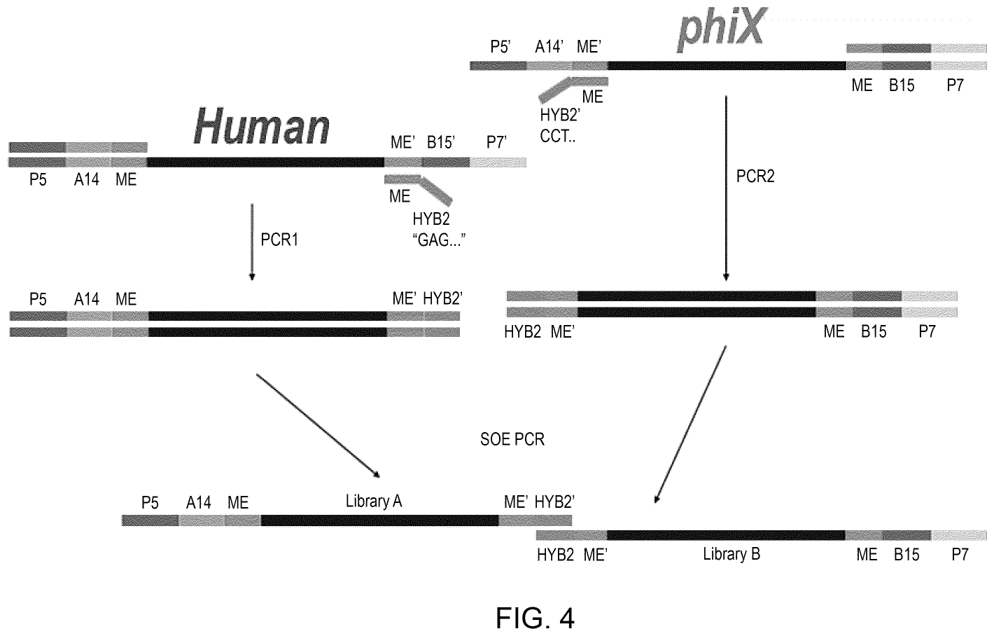

In some embodiments, the library may be prepared using PCR stitching methods, such as (splicing by) overlap extension PCR (also known as OE-PCR or SOE-PCR), as described in more detail in e.g. Higuchi et al. (Nucleic Acids Res., 1988, vol. 16, pp. 7351-7367), which is incorporated herein by reference. This procedure may be used, for example, for preparing templates including concatenated polynucleotide sequences comprising a first portion and a second portion, wherein the first portion and the second portion are different polynucleotide sequences (e.g. genetically unrelated, and/or obtained from different sources). A representative process for conducting PCR stitching for a human and PhiX library is shown in FIG. 4.

As used herein, the term “genetically unrelated” refers to portions which are not related in the sense of being any two of the group consisting of: forward strands, reverse strands, forward complement strands, and reverse complement strands. However, the “genetically unrelated” sequences could be different fragment sequences which are derived from the same source, but are different fragments from that source (e.g. from the same fragmented library preparation process). This includes sequences that can be overlapping in sequence (but not identical in sequence).

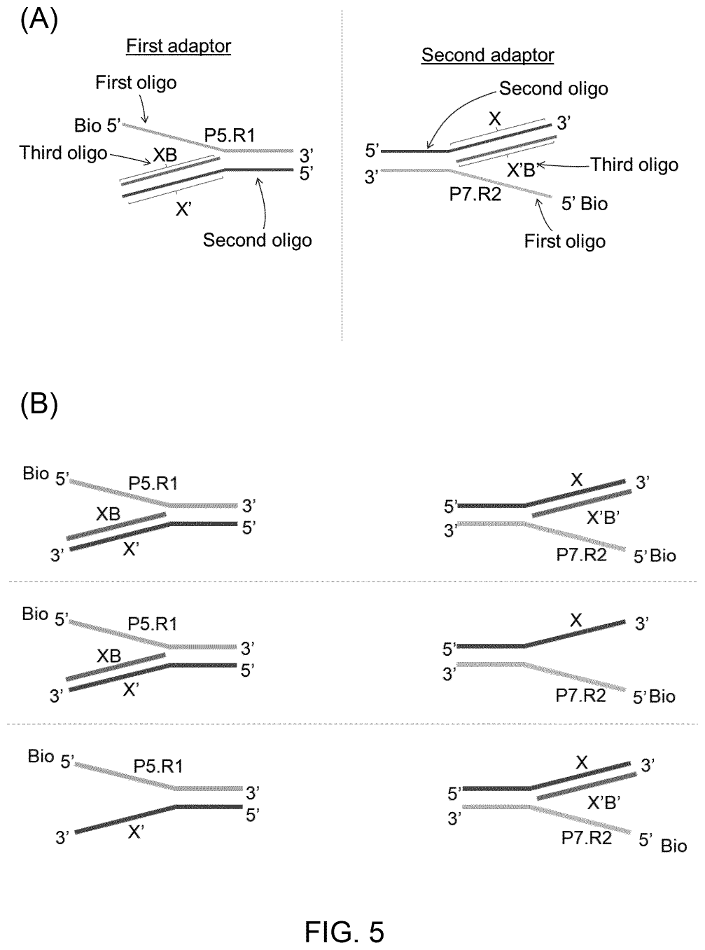

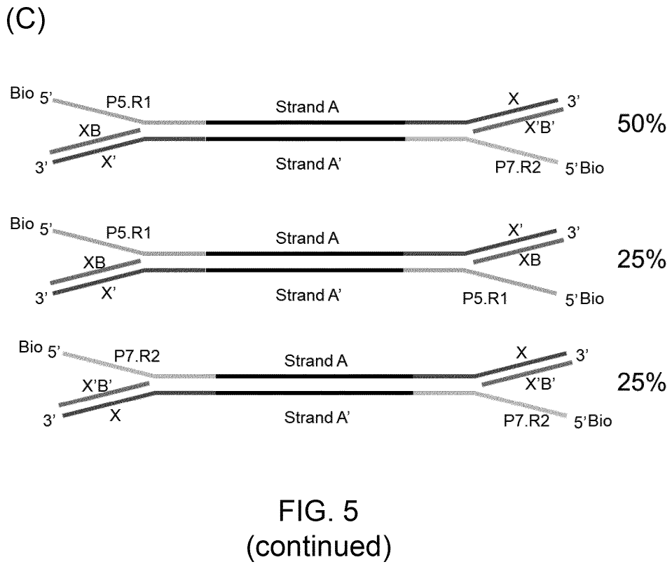

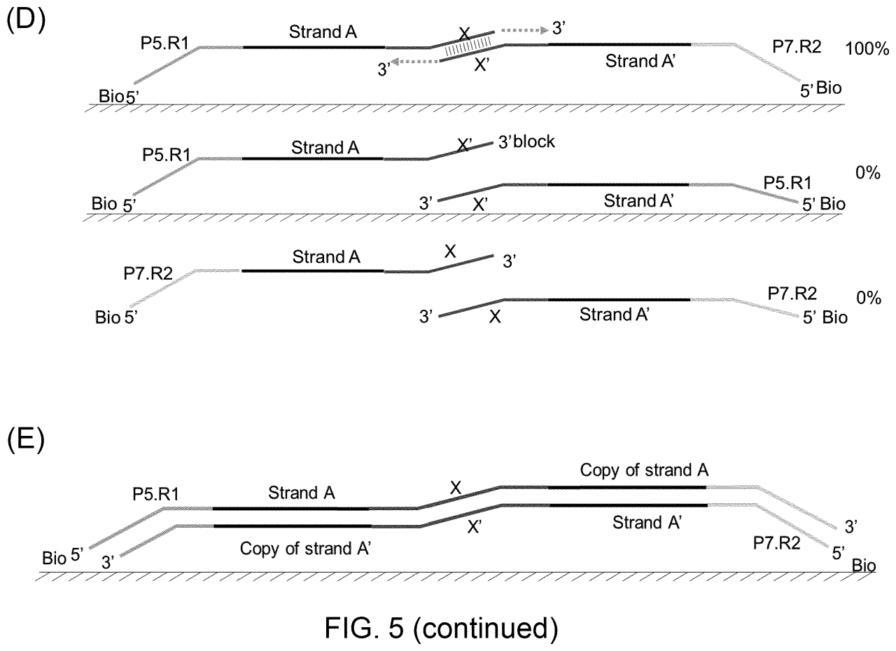

In some embodiments, the library may be prepared by using a tandem insert method described in more detail in e.g. WO 2022/087150, which is incorporated herein by reference. This procedure may be used, for example, for preparing templates comprising concatenated polynucleotide sequences comprising a first portion and a second portion, wherein the first portion is a forward strand of the template, and the second portion is a reverse complement strand of the template (or alternatively, wherein the first portion is a reverse strand of the template, and the second portion is a forward complement strand of the template). Such libraries may also be referred to as cross-tandem inserts. A representative process for conducting a tandem insert method is shown in FIG. 5(A) to 5(E).

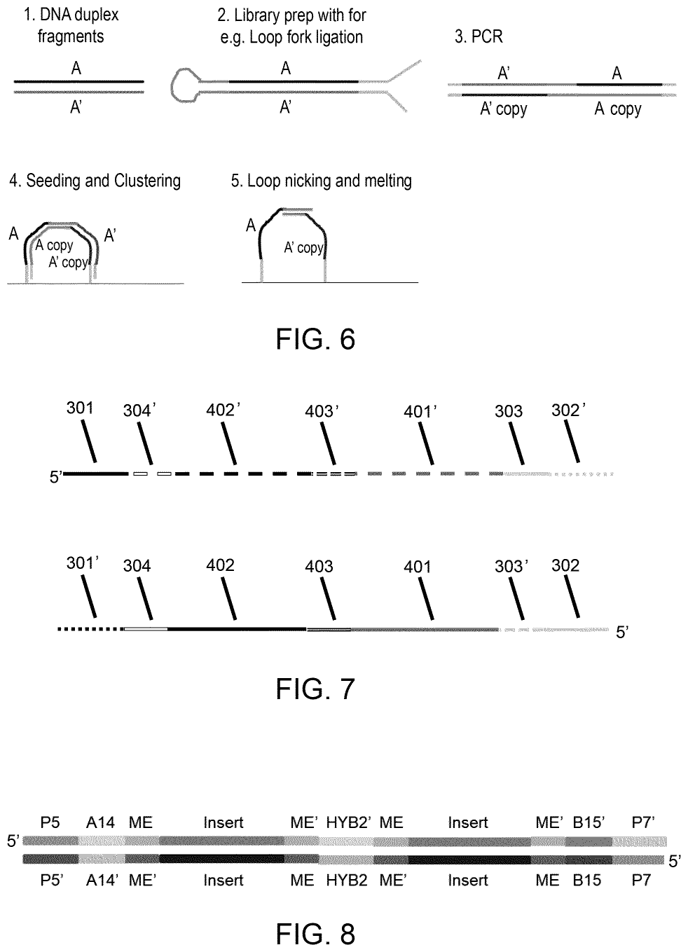

In some embodiments, the library may be prepared using a loop fork method, which is described below. This procedure may be used, for example, for preparing templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template). This procedure may also be used, for example, for preparing templates comprising concatenated polynucleotide sequences comprising a first portion and a second portion, wherein the first portion is a forward strand of the template, and the second portion is a reverse strand of the template. Such libraries may also be referred to as self-tandem inserts. A representative process for conducting a loop fork method is shown in FIG. 6.

Starting from a double-stranded polynucleotide sequence comprising a forward strand of the sequence and a reverse strand of the sequence, adaptors may be ligated to a first end of the sequence (e.g. using processes as described in more detail in e.g. WO 07/052006, or “tagmentation” methods as described above). A second end of the sequence (different from the first end) may be ligated to a loop, which connects the forward strand of the sequence and the reverse strand of the sequence, thus generating a loop fork ligated polynucleotide sequence. Conducting PCR on the loop fork ligated polynucleotide sequence produces a new double-stranded polynucleotide sequence, one strand comprising the forward strand of the sequence and the reverse strand of the sequence, and the other strand comprising a forward complement strand of the sequence and a reverse complement strand of the sequence. The library is now ready for seeding, clustering and amplification.

As will be described later, during clustering and amplification, further processes may be used to generate templates including a first polynucleotide sequence comprising a first portion and a second polynucleotide sequence comprising a second portion, wherein the first portion is a forward strand of the template, and the second portion is a reverse complement strand of the template (or alternatively, wherein the first portion is a reverse strand of the template, and the second portion is a forward complement strand of the template).

The processes described above in relation to PCR stitching, tandem insert methods, and loop fork methods generate libraries that have concatenated polynucleotides.

Thus, one strand of a concatenated polynucleotide within a polynucleotide library may comprise, in a 5′ to 3′ direction, a second primer-binding complement sequence 302 (e.g. P7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′) (FIGS. 7 and 8—bottom strand).

Although not shown in FIGS. 7 and 8, the strand may further comprise one or more index sequences. As such, a first index sequence (e.g. i7) may be provided between the second primer-binding complement sequence 302 (e.g. P7) and the first terminal sequencing primer binding site complement 303′ (e.g. B15-ME; or if ME is not present, then B15). Separately, or in addition, a second index complement sequence (e.g. i5′) may be provided between the second terminal sequencing primer binding site 304 (e.g. ME′-A14′) and the first primer-binding sequence 301′ (e.g. P5′). Thus, in some embodiments, one strand of a polynucleotide within a polynucleotide library may comprise, in a 5′ to 3′ direction, a second primer-binding complement sequence 302 (e.g. P7), a first index sequence (e.g. i7), a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), a second index complement sequence (e.g. i5′), and a first primer-binding sequence 301′ (e.g. P5′)

Another strand of a concatenated polynucleotide within a polynucleotide library may comprise, in a 5′ to 3′ direction, a first primer-binding complement sequence 301 (e.g. P5), a second terminal sequencing primer binding site complement 304′ (e.g. A14-ME; or if ME is not present, then A14), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), and a second primer-binding sequence 302′ (e.g. P7′) (FIGS. 7 and 8—top strand).

Although not shown in FIGS. 7 and 8, the another strand may further comprise one or more index sequences. As such, a second index sequence (e.g. i5) may be provided between the first primer-binding complement sequence 301 (e.g. P5) and the second terminal sequencing primer binding site complement 304′ (e.g. A14-ME; or if ME is not present, then A14). Separately, or in addition, a first index complement sequence (e.g. i7′) may be provided between the first terminal sequencing primer binding site 303 (e.g. ME′-B15′; or if ME′ is not present, then B15′) and the second primer-binding sequence 302′ (e.g. P7′). Thus, in some embodiments, another strand of a polynucleotide within a polynucleotide library may comprise, in a 5′ to 3′ direction, a first primer-binding complement sequence 301 (e.g. P5), a second index sequence (e.g. i5), a second terminal sequencing primer binding site complement 304′ (e.g. A14-ME; or if ME is not present, then A14).), a second insert complement sequence 402′, a hybridisation sequence 403′ (e.g. ME′-HYB2′-ME; or if ME′ and ME are not present, then HYB2′), a first insert complement sequence 401′, a first terminal sequencing primer binding site 303 (e.g. ME′-B15′; or if ME′ is not present, then B15′), a first index complement sequence (e.g. i7′), and a second primer-binding sequence 302′ (e.g. P7′).

As described herein, the first insert sequence 401 and the second insert sequence 402 may comprise different types of library sequences.

In one embodiment, the first insert sequence 401 may be different to the second insert sequence 402 (e.g. genetically unrelated, and/or obtained from different sources), for example where the library is prepared using PCR stitching.

In another embodiment, the first insert sequence 401 may comprise a forward strand of the sequence 101, and the second insert sequence may comprise a reverse complement strand of the sequence 102′ (or the first insert sequence 401 may comprise a reverse strand of the sequence 102, and the second insert sequence 402 may comprise a forward complement strand of the sequence 101′), for example where the library is prepared using a tandem insert method.

In another embodiment, the first insert sequence 401 may comprise a forward strand of the sequence 101, and the second insert sequence 402 may comprise a reverse strand of the sequence 102 (or the first insert sequence 401 may comprise a forward complement strand of the sequence 101′, and the second insert sequence 402 may comprise a reverse complement strand of the sequence 102′), for example where the library is prepared using a loop fork method.

As will be understood by the skilled person, a double-stranded nucleic acid will typically be formed from two complementary polynucleotide strands comprised of deoxyribonucleotides or ribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic acid.

A sequence comprising at least a primer-binding sequence (a primer-binding sequence and a sequencing primer binding site, in another aspect, a combination of a primer-binding sequence, an index sequence and a sequencing primer binding site) may be referred to herein as an adaptor sequence, and an insert (or inserts in concatenated strands) is flanked by a 5′ adaptor sequence and a 3′ adaptor sequence. The primer-binding sequence may also comprise a sequencing primer for the index read.

As used herein, an “adaptor” refers to a sequence that comprises a short sequence-specific oligonucleotide that is ligated to the 5′ and 3′ ends of each DNA (or RNA) fragment in a sequencing library as part of library preparation. The adaptor sequence may further comprise non-peptide linkers.

In a further embodiment, the P5′ and P7′ primer-binding sequences are complementary to short primer sequences (or lawn primers) present on the surface of a flow cell. Binding of P5′ and P7′ to their complements (P5 and P7) on—for example—the surface of the flow cell, permits nucleic acid amplification. As used herein “′” denotes the complementary strand.

The primer-binding sequences in the adaptor which permit hybridisation to amplification primers (e.g. lawn primers) will typically be around 20-40 nucleotides in length, although the invention is not limited to sequences of this length. The precise identity of the amplification primers (e.g. lawn primers), and hence the cognate sequences in the adaptors, are generally not material to the invention, as long as the primer-binding sequences are able to interact with the amplification primers in order to direct PCR amplification. The sequence of the amplification primers may be specific for a particular target nucleic acid that it is desired to amplify, but in other embodiments these sequences may be “universal” primer sequences which enable amplification of any target nucleic acid of known or unknown sequence which has been modified to enable amplification with the universal primers. The criteria for design of PCR primers are generally well known to those of ordinary skill in the art.

The index sequences (also known as a barcode or tag sequence) are unique short DNA (or RNA) sequences that are added to each DNA (or RNA) fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05/068656, whose contents are incorporated herein by reference in their entirety. The tag can be read at the end of the first read, or equally at the end of the second read, for example using a sequencing primer complementary to the strand marked P7. The invention is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example WO 2008/093098, which is incorporated herein by reference. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing primer binding sites are sequencing and/or index primer binding sites and indicate the starting point of the sequencing read. During the sequencing process, a sequencing primer anneals (i.e. hybridises) to at least a portion of the sequencing primer binding site on the template strand. The polymerase enzyme binds to this site and incorporates complementary nucleotides base by base into the growing opposite strand.

In concatenated strands, the hybridisation sequence (or the hybridisation sequence complement) may comprise an internal sequencing primer binding site. In other words, an internal sequencing primer binding site may form part of the hybridisation sequence. For example, ME′-HYB2 (or ME′-HYB2′) may act as an internal sequencing primer binding site to which a sequencing primer can bind. Alternatively, the hybridisation sequence may be an internal sequencing primer binding site. For example, HYB2 (or HYB2′) may act as an internal sequencing primer binding site to which a sequencing primer can bind. Accordingly, we may refer to the hybridisation site herein as comprising a second sequencing primer binding site, or as a second sequencing primer binding site.

### Cluster Generation and Amplification

Once a double stranded nucleic acid library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 4th Ed, Cold Spring Harbor Laboratory Press, Cold Spring Harbor Laboratory Press, NY; Current Protocols, eds Ausubel et al). In one embodiment, chemical denaturation may be used.

Following denaturation, a single-stranded library may be contacted in free solution onto a solid support comprising surface capture moieties (for example P5 and P7 lawn primers).

Thus, embodiments of the present invention may be performed on a solid support 200, such as a flowcell. However, in alternative embodiments, seeding and clustering can be conducted off-flowcell using other types of solid support.

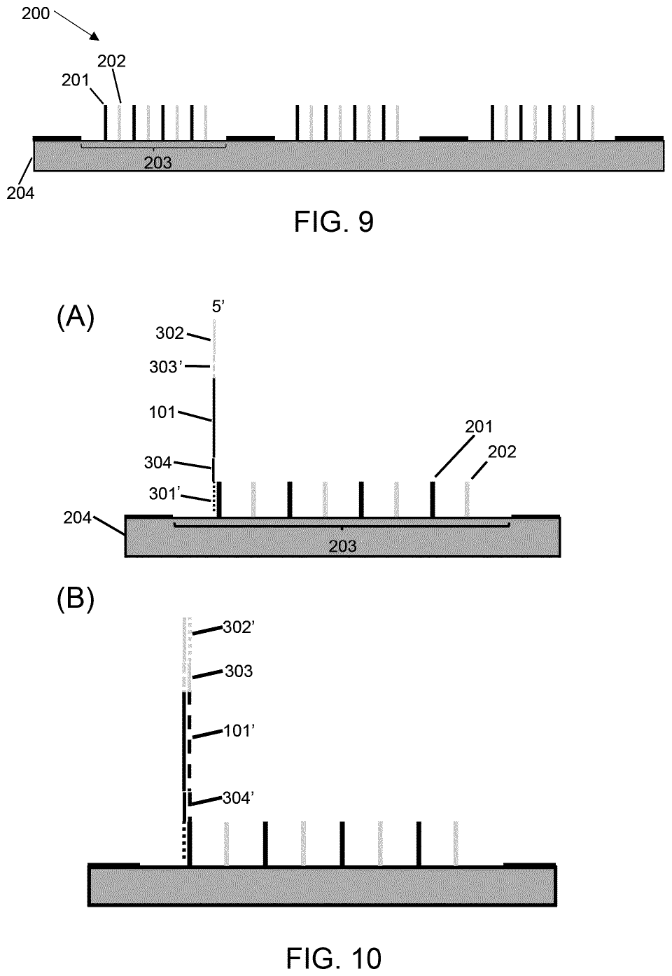

The solid support 200 may comprise a substrate 204. See FIG. 9. The substrate 204 comprises at least one well 203 (e.g. a nanowell), and typically comprises a plurality of wells 203 (e.g. a plurality of nanowells).

In one embodiment, the solid support comprises at least one first immobilised primer and at least one second immobilised primer.

Thus, each well 203 may comprise at least one first immobilised primer 201, and typically may comprise a plurality of first immobilised primers 201. In addition, each well 203 may comprise at least one second immobilised primer 202, and typically may comprise a plurality of second immobilised primers 202. Thus, each well 203 may comprise at least one first immobilised primer 201 and at least one second immobilised primer 202, and typically may comprise a plurality of first immobilised primers 201 and a plurality of second immobilised primers 202.

The first immobilised primer 201 may be attached via a 5′-end of its polynucleotide chain to the solid support 200. When extension occurs from first immobilised primer 201, the extension may be in a direction away from the solid support 200.

The second immobilised primer 202 may be attached via a 5′-end of its polynucleotide chain to the solid support 200. When extension occurs from second immobilised primer 202, the extension may be in a direction away from the solid support 200.

The first immobilised primer 201 may be different to the second immobilised primer 202 and/or a complement of the second immobilised primer 202. The second immobilised primer 202 may be different to the first immobilised primer 201 and/or a complement of the first immobilised primer 201.

The (or each of the) first immobilised primer(s) 201 may comprise a sequence as defined in SEQ ID NO: 1 or 5, or a variant or fragment thereof. The second immobilised primer(s) 202 may comprise a sequence as defined in SEQ ID NO: 2, or a variant or fragment thereof.

By way of brief example, following attachment of the P5 and P7 primers to the solid support, the solid support may be contacted with the template to be amplified under conditions which permit hybridisation (or annealing—such terms may be used interchangeably) between the template and the immobilised primers. The template is usually added in free solution under suitable hybridisation conditions, which will be apparent to the skilled reader. Typically, hybridisation conditions are, for example, 5×SSC at 40° C. However, other temperatures may be used during hybridisation, for example about 50° C. to about 75° C., about 55° C. to about 70° C., or about 60° C. to about 65° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e. either P5′ or P7′) which is capable of bridging to the second primer molecule immobilised on the solid support and binding. Further rounds of amplification (analogous to a standard PCR reaction) leads to the formation of clusters or colonies of template molecules bound to the solid support. This is called clustering.

Thus, solid-phase amplification by either a method analogous to that of WO 98/44151 or that of WO 00/18957 (the contents of which are incorporated herein in their entirety by reference) will result in production of a clustered array comprised of colonies of “bridged” amplification products. This process is known as bridge amplification. Both strands of the amplification products will be immobilised on the solid support at or near the 5′ end, this attachment being derived from the original attachment of the amplification primers. Typically, the amplification products within each colony will be derived from amplification of a single template molecule. Other amplification procedures may be used, and will be known to the skilled person. For example, amplification may be isothermal amplification using a strand displacement polymerase; or may be exclusion amplification as described in WO 2013/188582. Further information on amplification can be found in WO 02/06456 and WO 07/107710, the contents of which are incorporated herein in their entirety by reference.

Through such approaches, a cluster of template molecules is formed, comprising copies of a template strand and copies of the complement of the template strand.

In some cases, to facilitate sequencing, one set of strands (either the original template strands or the complement strands thereof) may be removed from the solid support leaving either the original template strands or the complement strands. Suitable methods for removing such strands are described in more detail in application number WO 07/010251, the contents of which are incorporated herein by reference in their entirety.

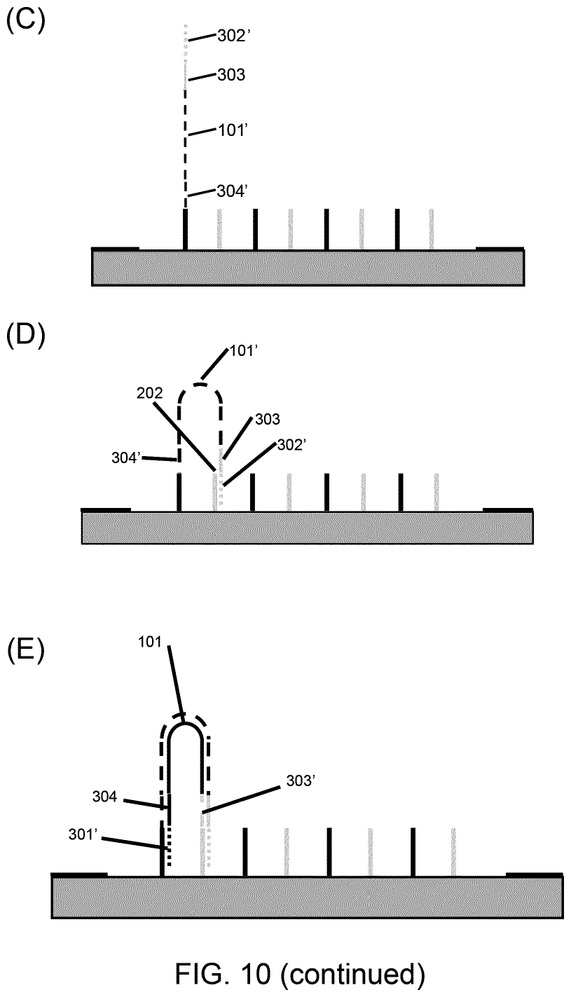

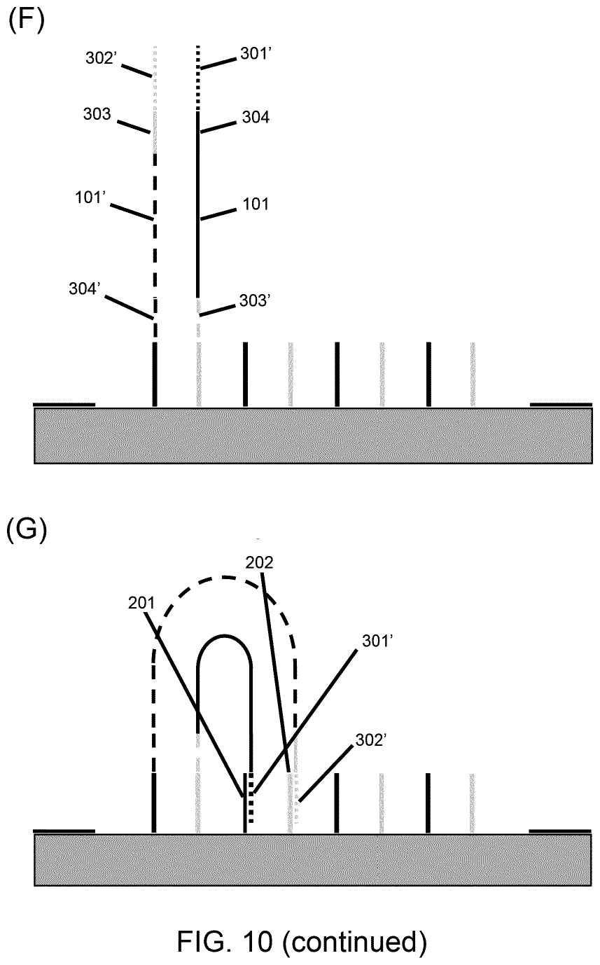

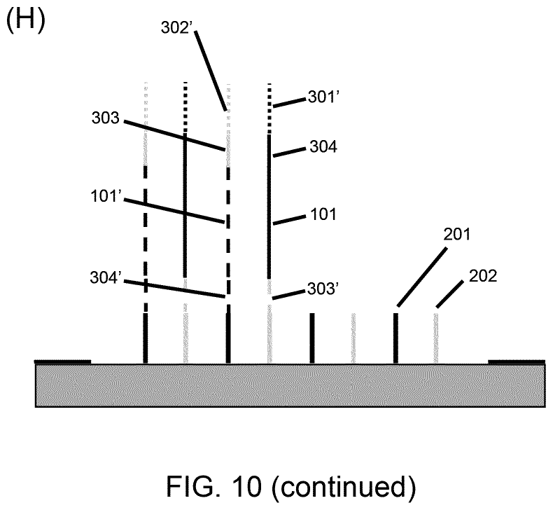

The steps of cluster generation and amplification for templates including a concatenated polynucleotide sequence comprising a first portion and a second portion, as well as templates including a first polynucleotide sequence comprising a first portion and a second polynucleotide sequence comprising a second portion, are illustrated below and in FIG. 10.

In cases where single (concatenated) polynucleotide strands are used, each polynucleotide sequence may be attached (via the 5′-end of the (concatenated) polynucleotide sequence) to a first immobilised primer. Each polynucleotide sequence may comprise a second adaptor sequence, wherein the second adaptor comprises a portion, which is substantially complementary to the second immobilised primer (or is substantially complementary to the second immobilised primer). The second adaptor sequence may be at a 3′-end of the (concatenated) polynucleotide sequence.

In cases where (separate) polynucleotide strands are used, each first polynucleotide sequence may be attached (via the 5′-end of the first polynucleotide sequence) to a first immobilised primer, and wherein each second polynucleotide sequence is attached (via the 5′-end of the second polynucleotide sequence) to a second immobilised primer. Each first polynucleotide sequence may comprise a second adaptor sequence, wherein the second adaptor sequence comprises a portion, which is substantially complementary to the second immobilised primer (or is substantially complementary to the second immobilised primer). The second adaptor sequence may be at a 3′-end of the first polynucleotide sequence. Each second polynucleotide sequence may comprise a first adaptor sequence, wherein the first adaptor sequence comprises a portion, which is substantially complementary to the first immobilised primer (or is substantially complementary to the first immobilised primer). The first adaptor sequence may be at a 3′-end of the second polynucleotide sequence.

In an embodiment, a solution comprising a polynucleotide library prepared by ligating adaptor sequences to double-stranded polynucleotide sequences as described above may be flown across a flowcell.

A particular polynucleotide strand from the polynucleotide library to be sequenced comprising, in a 5′ to 3′ direction, a second primer-binding complement sequence 302 (e.g. P7), a first terminal binding site complement 303′ (e.g. SBS12), a forward strand of the sequence 101, a second terminal sequencing primer binding site 304 (e.g. SBS3′) and a first primer-binding sequence 301′ (e.g. P5′), may anneal (via the first primer-binding sequence 301′) to the first immobilised primer 201 (e.g. P5 lawn primer) located within a particular well 203 (FIG. 10A).

The polynucleotide library may comprise other polynucleotide strands with different forward strands of the sequence 101. Such other polynucleotide strands may anneal to corresponding first immobilised primers 201 (e.g. P5 lawn primers) in different wells 203, thus enabling parallel processing of the various different strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, a second terminal sequencing primer binding site complement 304′ (e.g. SBS3), a forward strand of the template 101′ (which represents a type of “first portion”), a first terminal sequencing primer binding site 303 (which represents a type of “first sequencing primer binding site”) (e.g. SBS12′), and a second primer-binding sequence 302′ (e.g. P7′) (FIG. 10B). Such a process may utilise an appropriate polymerase, such as a DNA or RNA polymerase.

If the polynucleotides in the library comprise index sequences, then corresponding index sequences are also produced in the template.

The polynucleotide strand from the polynucleotide library may then be dehybridised and washed away, leaving a template strand attached to the first immobilised primer 201 (e.g. P5 lawn primer) (FIG. 10C).

The second primer-binding sequence 302′ (e.g. P7′) on the template strand may then anneal to a second immobilised primer 202 (e.g. P7 lawn primer) located within the well 203. This forms a “bridge” (FIG. 10D).

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, a first terminal sequencing primer binding site complement 303′ (e.g. SBS12), a forward complement strand of the template 101 (which represents a type of “second portion”), a second terminal sequencing primer binding site 304 (which represents a type of “second sequencing primer binding site”) (e.g. SBS3′), and a first primer-binding sequence 301′ (e.g. P5′) (FIG. 10E). Again, such a process may utilise a suitable polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 10F).

A subsequent bridge amplification cycle can then lead to amplification of the strand attached to the first immobilised primer 201 (e.g. P5 lawn primer) and the strand attached to the second immobilised primer 202 (e.g. P7 lawn primer). Similar to FIG. 10D, the second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203 (FIG. 10G).

Completion of bridge amplification and dehybridisation may then provide an amplified (duoclonal) cluster, thus providing a plurality of first polynucleotide sequences comprising the forward strand of the template 101′ (i.e. “first portions”), and a plurality of second polynucleotide sequences comprising the forward complement strand of the template 101 (i.e. “second portions”) (FIG. 10H).

If desired, further bridge amplification cycles may be conducted to increase the number of first polynucleotide sequences and second polynucleotide sequences within the well 203.

In this particular example, the “first portion” corresponds with the forward strand of the template 101′, and the “second portion” corresponds with the forward complement strand of the template 101.

However, other set-ups may be obtained by changing the library used. For example, by using a loop fork method to prepare a library, a portion at or close to the loop (or the loop complement) may be cleaved (e.g. by nicking). In these cases, the loop may comprise a cleavage site (e.g. a restriction recognition site, a cleavable linker, a modified nucleotide, or the like). By conducting cleavage at the loop, it is possible to produce a well 203, where the “first portion” corresponds with a forward strand of the template, and the “second portion” corresponds with a reverse complement strand of the template. As such, different types of strands for the “first portions” and “second portions” may be prepared for templates including a first polynucleotide sequence comprising a first portion and a second polynucleotide sequence comprising a second portion.

In an embodiment, a solution comprising a polynucleotide library prepared by PCR stitching, a tandem insert method or a loop fork method as described above may be flowed across a flowcell.

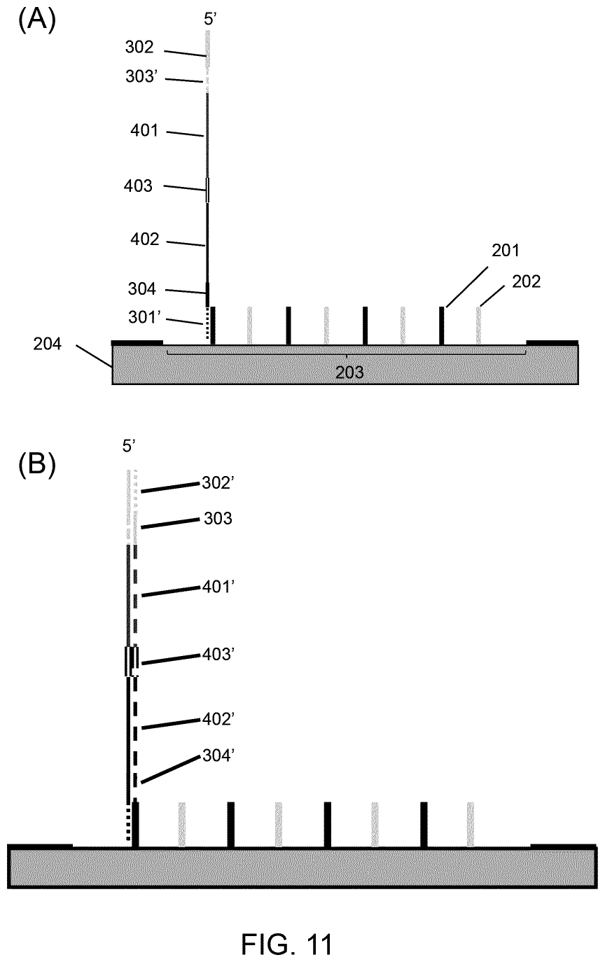

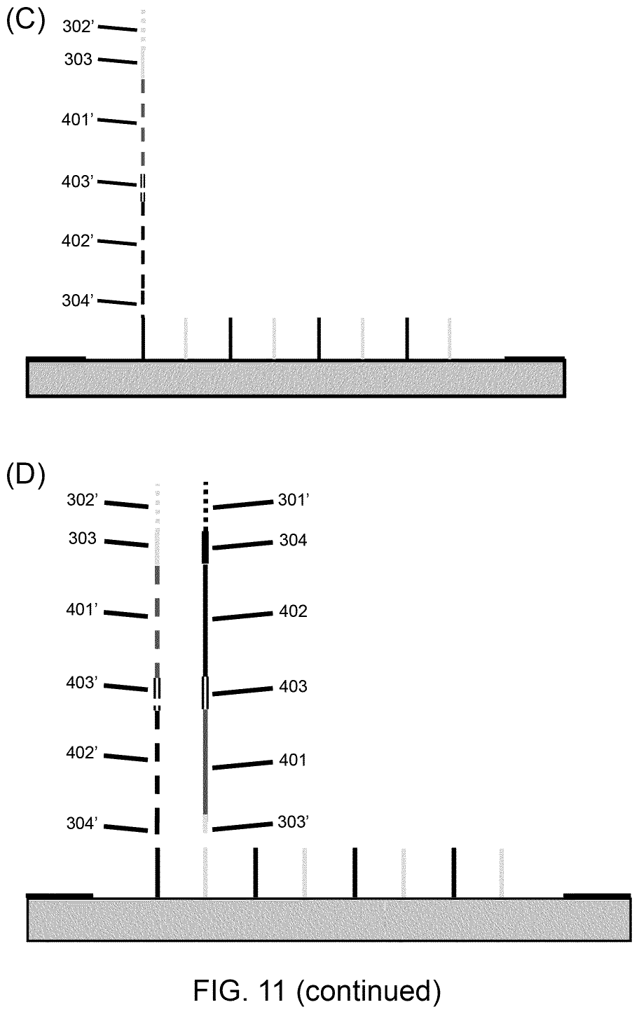

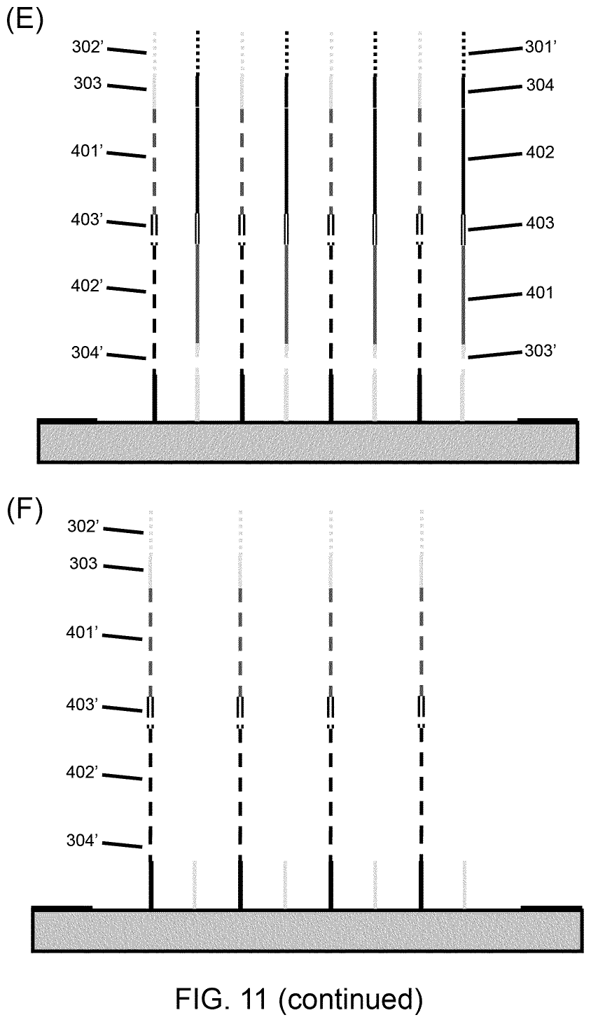

A particular concatenated polynucleotide strand from the polynucleotide library to be sequenced comprising, in a 5′ to 3′ direction, a second primer-binding complement sequence 302 (e.g. P7), a first terminal sequencing primer binding site complement 303′ (e.g. B15-ME), a first insert sequence 401, a hybridisation complement sequence 403 (e.g. ME′-HYB2-ME), a second insert sequence 402, a second terminal sequencing primer binding site 304 (e.g. ME′-A14′), and a first primer-binding sequence 301′ (e.g. P5′), may anneal (via the first primer-binding sequence 301′) to the first immobilised primer 201 (e.g. P5 lawn primer) located within a particular well 203 (FIG. 11A).

The polynucleotide library may comprise other concatenated polynucleotide strands with different first insert sequences 401 and second insert sequences 402. Such other polynucleotide strands may anneal to corresponding first immobilised primers 201 (e.g. P5 lawn primers) in different wells 203, thus enabling parallel processing of the various different concatenated strands within the polynucleotide library.

A new polynucleotide strand may then be synthesised, extending from the first immobilised primer 201 (e.g. P5 lawn primer) in a direction away from the substrate 204. By using complementary base-pairing, this generates a template strand comprising, in a 5′ to 3′ direction, the first immobilised primer 201 (e.g. P5 lawn primer) which is attached to the solid support 200, a second terminal sequencing primer binding site complement 304′ (e.g. A14-ME; or if ME is not present, then A14), a second insert complement sequence 402′ (which represents a type of “second portion”), a hybridisation sequence 403′ (which comprises a type of “second sequencing primer binding site”) (e.g. ME′-HYB2′-ME; or if ME′ and ME are not present, then HYB2′), a first insert complement sequence 401′ (which represents a type of “first portion”), a first terminal sequencing primer binding site 303 (which represents a type of “first sequencing primer binding site”) (e.g. ME′-B15′; or if ME′ is not present, then B15′), and a second primer-binding sequence 302′ (e.g. P7′) (FIG. 11B). Such a process may utilise a polymerase, such as a DNA or RNA polymerase.

If the polynucleotides in the library comprise index sequences, then corresponding index sequences are also produced in the template.

The concatenated polynucleotide strand from the polynucleotide library may then be dehybridised and washed away, leaving a template strand attached to the first immobilised primer 201 (e.g. P5 lawn primer) (FIG. 11C).

The second primer-binding sequence 302′ (e.g. P7′) on the template strand may then anneal to a second immobilised primer 202 (e.g. P7 lawn primer) located within the well 203. This forms a “bridge” (not shown, but using a similar process as shown in FIG. 10D).

A new polynucleotide strand may then be synthesised by bridge amplification, extending from the second immobilised primer 202 (e.g. P7 lawn primer) (initially) in a direction away from the substrate 204. By using complementary base-pairing, this generates a template strand comprising, in a 5′ to 3′ direction, the second immobilised primer 202 (e.g. P7 lawn primer) which is attached to the solid support 200, a first terminal sequencing primer binding site complement 303′ (e.g. B15-ME; or if ME is not present, then B15), a first insert sequence 401, a hybridisation complement sequence 403 (e.g. ME′-HYB2-ME; or if ME′ and ME are not present, then HYB2), a second insert sequence 402, a second terminal sequencing primer binding site 304 (e.g. ME′-A14′; or if ME′ is not present, then A14′), and a first primer-binding sequence 301′ (e.g. P5′) (not shown, but using a similar process as shown in FIG. 10E). Again, such a process may utilise a polymerase, such as a DNA or RNA polymerase.

The strand attached to the second immobilised primer 202 (e.g. P7 lawn primer) may then be dehybridised from the strand attached to the first immobilised primer 201 (e.g. P5 lawn primer) (FIG. 11D).

A subsequent bridge amplification cycle can then lead to amplification of the strand attached to the first immobilised primer 201 (e.g. P5 lawn primer) and the strand attached to the second immobilised primer 202 (e.g. P7 lawn primer). Similar to FIG. 10D, the second primer-binding sequence 302′ (e.g. P7′) on the template strand attached to the first immobilised primer 201 (e.g. P5 lawn primer) may then anneal to another second immobilised primer 202 (e.g. P7 lawn primer) located within the well 203. In a similar fashion, the first primer-binding sequence 301′ (e.g. P5′) on the template strand attached to the second immobilised primer 202 (e.g. P7 lawn primer) may then anneal to another first immobilised primer 201 (e.g. P5 lawn primer) located within the well 203 (not shown, but using a similar process as shown in FIG. 10G).

Completion of bridge amplification and dehybridisation may then provide an amplified cluster, thus providing a plurality of concatenated polynucleotide sequences comprising a first insert complement sequence 401′ (i.e. “first portions”) and a second insert complement sequence 402′ (i.e. second portions”), as well as a plurality of concatenated polynucleotide sequences comprising a first insert sequence 401 and a second insert sequence 402 (FIG. 11E).

If desired, further bridge amplification cycles may be conducted to increase the number of first polynucleotide sequences and second polynucleotide sequences within the well 203.

In one aspect, before sequencing, one group of strands (either the group of template polynucleotides, or the group of template complement polynucleotides thereof) is removed from the solid support to form a (monoclonal) cluster, leaving either the templates or the template complements (FIG. 11F).

The methods for clustering and amplification described above generally relate to conducting non-selective amplification. However, methods of the present invention relating to selective processing may comprise conducting selective amplification, which is described in further detail below under selective processing.

### Sequencing

As described herein, the template provides information (e.g. identification of the genetic sequence, identification of epigenetic modifications) on the original target polynucleotide sequence. For example, a sequencing process (e.g. a sequencing-by-synthesis or sequencing-by-ligation process) may reproduce information that was present in the original target polynucleotide sequence, by using complementary base pairing.

In one embodiment, sequencing may be carried out using any suitable “sequencing-by-synthesis” technique, wherein nucleotides are added successively in cycles to the free 3′ hydroxyl group, resulting in synthesis of a polynucleotide chain in the 5′ to 3′ direction. In one embodiment, the nature of the nucleotide added is determined after each addition. One particular sequencing method relies on the use of modified nucleotides that can act as reversible chain terminators. Such reversible chain terminators comprise removable 3′ blocking groups. Once such a modified nucleotide has been incorporated into the growing polynucleotide chain complementary to the region of the template being sequenced, there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the contents of which are incorporated herein by reference in their entirety. Alternatively, a separate reaction may be carried out containing each of the modified nucleotides added individually.

The modified nucleotides may carry a label to facilitate their detection. Such a label may be configured to emit a signal, such as an electromagnetic signal, or a (visible) light signal.

In a particular embodiment, the label is a fluorescent label (e.g. a dye). Thus, such a label may be configured to emit an electromagnetic signal, or a (visible) light signal. One method for detecting the fluorescently labelled nucleotides comprises using laser light of a wavelength specific for the labelled nucleotides, or the use of other suitable sources of illumination. The fluorescence from the label on an incorporated nucleotide may be detected by a CCD camera or other suitable detection means. Suitable detection means are described in PCT/US2007/007991, the contents of which are incorporated herein by reference in their entirety.

However, the detectable label need not be a fluorescent label. Any label can be used which allows the detection of the incorporation of the nucleotide into the DNA sequence.

Each cycle may involve simultaneous delivery of four different nucleotide types to the array of template molecules. Alternatively, different nucleotide types can be added sequentially and an image of the array of template molecules can be obtained between each addition step.

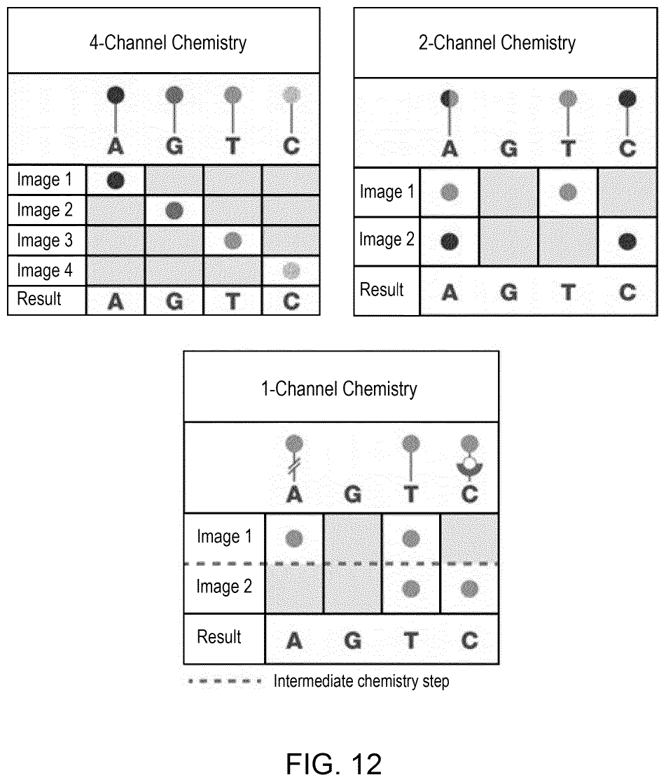

In some embodiments, each nucleotide type may have a (spectrally) distinct label. In other words, four channels may be used to detect four nucleobases (also known as 4-channel chemistry) (FIG. 12—left). For example, a first nucleotide type (e.g. A) may include a first label (e.g. configured to emit a first wavelength, such as red light), a second nucleotide type (e.g. G) may include a second label (e.g. configured to emit a second wavelength, such as blue light), a third nucleotide type (e.g. T) may include a third label (e.g. configured to emit a third wavelength, such as green light), and a fourth nucleotide type (e.g. C) may include a fourth label (e.g. configured to emit a fourth wavelength, such as yellow light). Four images can then be obtained, each using a detection channel that is selective for one of the four different labels. For example, the first nucleotide type (e.g. A) may be detected in a first channel (e.g. configured to detect the first wavelength, such as red light), the second nucleotide type (e.g. G) may be detected in a second channel (e.g. configured to detect the second wavelength, such as blue light), the third nucleotide type (e.g. T) may be detected in a third channel (e.g. configured to detect the third wavelength, such as green light), and the fourth nucleotide type (e.g. C) may be detected in a fourth channel (e.g. configured to detect the fourth wavelength, such as yellow light). Although specific pairings of bases to signal types (e.g. wavelengths) are described above, different signal types (e.g. wavelengths) and/or permutations may also be used.

In some embodiments, detection of each nucleotide type may be conducted using fewer than four different labels. For example, sequencing-by-synthesis may be performed using methods and systems described in US 2013/0079232, which is incorporated herein by reference.

Thus, in some embodiments, two channels may be used to detect four nucleobases (also known as 2-channel chemistry) (FIG. 12—middle). For example, a first nucleotide type (e.g. A) may include a first label (e.g. configured to emit a first wavelength, such as green light) and a second label (e.g. configured to emit a second wavelength, such as red light), a second nucleotide type (e.g. G) may not include the first label and may not include the second label, a third nucleotide type (e.g. T) may include the first label (e.g. configured to emit the first wavelength, such as green light) and may not include the second label, and a fourth nucleotide type (e.g. C) may not include the first label and may include the second label (e.g. configured to emit the second wavelength, such as red light). Two images can then be obtained, using detection channels for the first label and the second label. For example, the first nucleotide type (e.g. A) may be detected in both a first channel (e.g. configured to detect the first wavelength, such as red light) and a second channel (e.g. configured to detect the second wavelength, such as green light), the second nucleotide type (e.g. G) may not be detected in the first channel and may not be detected in the second channel, the third nucleotide type (e.g. T) may be detected in the first channel (e.g. configured to detect the first wavelength, such as red light) and may not be detected in the second channel, and the fourth nucleotide type (e.g. C) may not be detected in the first channel and may be detected in the second channel (e.g. configured to detect the second wavelength, such as green light). Although specific pairings of bases to signal types (e.g. wavelengths) and/or combinations of channels are described above, different signal types (e.g. wavelengths) and/or permutations may also be used.

In some embodiments, one channel may be used to detect four nucleobases (also known as 1-channel chemistry) (FIG. 12—right). For example, a first nucleotide type (e.g. A) may include a cleavable label (e.g. configured to emit a wavelength, such as green light), a second nucleotide type (e.g. G) may not include a label, a third nucleotide type (e.g. T) may include a non-cleavable label (e.g. configured to emit the wavelength, such as green light), and a fourth nucleotide type (e.g. C) may include a label-accepting site which does not include the label. A first image can then be obtained, and a subsequent treatment carried out to cleave the label attached to the first nucleotide type, and to attach the label to the label-accepting site on the fourth nucleotide type. A second image may then be obtained. For example, the first nucleotide type (e.g. A) may be detected in a channel (e.g. configured to detect the wavelength, such as green light) in the first image and not detected in the channel in the second image, the second nucleotide type (e.g. G) may not be detected in the channel in the first image and may not be detected in the channel in the second image, the third nucleotide type (e.g. T) may be detected in the channel (e.g. configured to detect the wavelength, such as green light) in the first image and may be detected in the channel (e.g. configured to detect the wavelength, such as green light) in the second image, and the fourth nucleotide type (e.g. C) may not be detected in the channel in the first image and may be detected in the channel in the second image (e.g. configured to detect the wavelength, such as green light). Although specific pairings of bases to signal types (e.g. wavelengths) and/or combinations of images are described above, different signal types (e.g. wavelengths), images and/or permutations may also be used.

In one embodiment, the sequencing process comprises a first sequencing read and second sequencing read. The first sequencing read and the second sequencing read may be conducted concurrently. In other words, the first sequencing read and the second sequencing read may be conducted at the same time.

The first sequencing read may comprise the binding of a first sequencing primer (also known as a read 1 sequencing primer) to the first sequencing primer binding site (e.g. first terminal sequencing primer binding site 303 in templates including a first polynucleotide sequence comprising a first portion and a second polynucleotide sequence comprising a second portion, or templates including a concatenated polynucleotide sequence comprising a first portion and a second portion). The second sequencing read may comprise the binding of a second sequencing primer (also known as a read 2 sequencing primer) to the second sequencing primer binding site (e.g. second terminal sequencing primer binding site 304 in templates including a first polynucleotide sequence comprising a first portion and a second polynucleotide sequence comprising a second portion, or a portion of hybridisation sequence 403′ in templates including a concatenated polynucleotide sequence comprising a first portion and a second portion).

This leads to sequencing of the first portion (e.g. forward strand of the template 101′ in templates including a first polynucleotide sequence comprising a first portion and a second polynucleotide sequence comprising a second portion, or first insert complement sequence 401′ in templates including a concatenated polynucleotide sequence comprising a first portion and a second portion) and the second portion (e.g. forward complement strand of the template 101 in templates including a first polynucleotide sequence comprising a first portion and a second polynucleotide sequence comprising a second portion, or second insert complement sequence 402′ in templates including a concatenated polynucleotide sequence comprising a first portion and a second portion).

Alternative methods of sequencing include sequencing by ligation, for example as described in U.S. Pat. No. 6,306,597 or WO 06/084132, the contents of which are incorporated herein by reference.

The methods for sequencing described above generally relate to conducting non-selective sequencing. However, methods of the present invention relating to selective processing may comprise conducting selective sequencing, which is described in further detail below under selective processing.

### Data Analysis Using 16 QaM

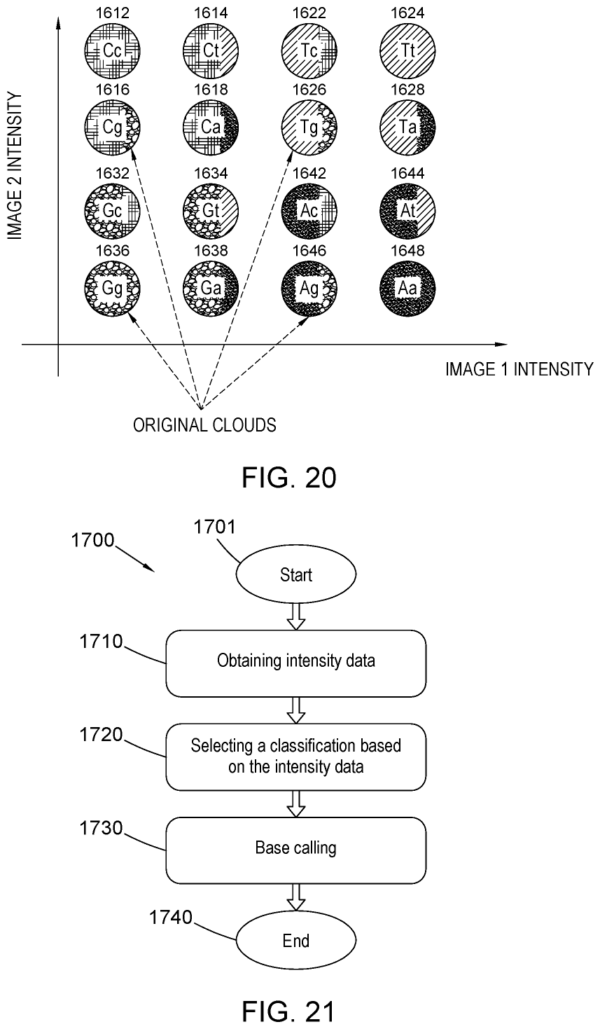

FIG. 20 is a scatter plot showing an example of sixteen distributions of signals generated by polynucleotide sequences disclosed herein.

The scatter plot of FIG. 20 shows sixteen distributions (or bins) of intensity values from the combination of a brighter signal (i.e. a first signal as described herein) and a dimmer signal (i.e. a second signal as described herein); the two signals may be co-localized and may not be optically resolved as described above. The intensity values shown in FIG. 20 may be up to a scale or normalisation factor; the units of the intensity values may be arbitrary or relative (i.e., representing the ratio of the actual intensity to a reference intensity). The sum of the brighter signal generated by the first portions and the dimmer signal generated by the second portions results in a combined signal. The combined signal may be captured by a first optical channel and a second optical channel. Since the brighter signal may be A, T, C or G, and the dimmer signal may be A, T, C or G, there are sixteen possibilities for the combined signal, corresponding to sixteen distinguishable patterns when optically captured. That is, each of the sixteen possibilities corresponds to a bin shown in FIG. 20. The computer system can map the combined signal generated into one of the sixteen bins, and thus determine the added nucleobase at the first portion and the added nucleobase at the second portion, respectively.

For example, when the combined signal is mapped to bin 1612 for a base calling cycle, the computer processor base calls both the added nucleobase at the first portion and the added nucleobase at the second portion as C. When the combined signal is mapped to bin 1614 for the base calling cycle, the processor base calls the added nucleobase at the first portion as C and the added nucleobase at the second portion as T. When the combined signal is mapped to bin 1616 for the base calling cycle, the processor base calls the added nucleobase at the first portion as C and the added nucleobase at the second portion as G. When the combined signal is mapped to bin 1618 for the base calling cycle, the processor base calls the added nucleobase at the first portion as C and the added nucleobase at the second portion as A.

When the combined signal is mapped to bin 1622 for the base calling cycle, the processor base calls the added nucleobase at the first portion as T and the added nucleobase at the second portion as C. When the combined signal is mapped to bin 1624 for the base calling cycle, the processor base calls both the added nucleobase at the first portion and the added nucleobase at the second portion as T. When the combined signal is mapped to bin 1626 for the base calling cycle, the processor base calls the added nucleobase at the first portion as T and the added nucleobase at the second portion as G. When the combined signal is mapped to bin 1628 for the base calling cycle, the processor base calls the added nucleobase at the first portion as T and the added nucleobase at the second portion as A.

When the combined signal is mapped to bin 1632 for the base calling cycle, the processor base calls the added nucleobase at the first portion as G and the added nucleobase at the second portion as C. When the combined signal is mapped to bin 1634 for the base calling cycle, the processor base calls the added nucleobase at the first portion as G and the added nucleobase at the second portion as T. When the combined signal is mapped to bin 1636 for the base calling cycle, the processor base calls both the added nucleobase at the first portion and the added nucleobase at the second portion as G. When the combined signal is mapped to bin 1638 for the base calling cycle, the processor base calls the added nucleobase at the first portion as G and the added nucleobase at the second portion as A.

When the combined signal is mapped to bin 1642 for the base calling cycle, the processor base calls the added nucleobase at the first portion as A and the added nucleobase at the second portion as C. When the combined signal is mapped to bin 1644 for the base calling cycle, the processor base calls the added nucleobase at the first portion as A and the added nucleobase at the second portion as T. When the combined signal is mapped to bin 1646 for the base calling cycle, the processor base calls the added nucleobase at the first portion as A and the added nucleobase at the second portion as G. When the combined signal is mapped to bin 1648 for the base calling cycle, the processor base calls both the added nucleobase at the first portion and the added nucleobase at the second portion as A.

In this particular example, T is configured to emit a signal in both the IMAGE 1 channel and the IMAGE 2 channel, A is configured to emit a signal in the IMAGE 1 channel only, C is configured to emit a signal in the IMAGE 2 channel only, and G does not emit a signal in either channel. However, different permutations of nucleobases can be used to achieve the same effect by performing dye swaps. For example, A may be configured to emit a signal in both the IMAGE 1 channel and the IMAGE 2 channel, T may be configured to emit a signal in the IMAGE 1 channel only, C may be configured to emit a signal in the IMAGE 2 channel only, and G may be configured to not emit a signal in either channel.

Further details regarding performing base-calling based on a scatter plot having sixteen bins may be found in U.S. Patent Application Publication No. 2019/0212294, the disclosure of which is incorporated herein by reference.

FIG. 21 is a flow diagram showing a method 1700 of base calling according to the present disclosure. The described method allows for simultaneous sequencing of two (or more) portions (e.g. the first portion and the second portion) in a single sequencing run from a single combined signal obtained from the first portion and the second portion, thus requiring less sequencing reagent consumption and faster generation of data from both the first portion and the second portion. Further, the simplified method may reduce the number of workflow steps while producing the same yield as compared to existing next-generation sequencing methods. Thus, the simplified method may result in reduced sequencing runtime.

As shown in FIG. 21, the disclosed method 1700 may start from block 1701. The method may then move to block 1710.

At block 1710, intensity data is obtained. The intensity data includes first intensity data and second intensity data. The first intensity data comprises a combined intensity of a first signal component obtained based upon a respective first nucleobase of the first portion and a second signal component obtained based upon a respective second nucleobase of the second portion. Similarly, the second intensity data comprises a combined intensity of a third signal component obtained based upon the respective first nucleobase of the first portion and a fourth signal component obtained based upon the respective second nucleobase of the second portion.

As such, the first portion is capable of generating a first signal comprising a first signal component and a third signal component. The second portion is capable of generating a second signal comprising a second signal component and a fourth signal component.

As described above, the first portion and the second portion may be arranged on the solid support such that signals from the first portion and the second portion are detected by a single sensing portion and/or may comprise a single cluster such that first signals and second signals from each of the respective first portions and second portions cannot be spatially resolved.

In one example, obtaining the intensity data comprises selecting intensity data that corresponds to two (or more) different portions (e.g. the first portion and the second portion). In one example, intensity data is selected based upon a chastity score. A chastity score may be calculated as the ratio of the brightest base intensity divided by the sum of the brightest and second brightest base intensities. The desired chastity score may be different depending upon the expected intensity ratio of the light emissions associated with the different portions. As described above, it may be desired to produce clusters comprising the first portion and the second portion, which give rise to signals in a ratio of 2:1. In one example, high-quality data corresponding to two portions with an intensity ratio of 2:1 may have a chastity score of around 0.8 to 0.9.

After the intensity data has been obtained, the method may proceed to block 1720. In this step, one of a plurality of classifications is selected based on the intensity data. Each classification represents a possible combination of respective first and second nucleobases. In one example, the plurality of classifications comprises sixteen classifications as shown in FIG. 20, each representing a unique combination of first and second nucleobases. Where there are two portions, there are sixteen possible combinations of first and second nucleobases. Selecting the classification based on the first and second intensity data comprises selecting the classification based on the combined intensity of the first and second signal components and the combined intensity of the third and fourth signal components.

The method may then proceed to block 1730, where the respective first and second nucleobases are base called based on the classification selected in block 1720. The signals generated during a cycle of a sequencing are indicative of the identity of the nucleobase(s) added during sequencing (e.g. using sequencing-by-synthesis). It will be appreciated that there is a direct correspondence between the identity of the nucleobases that are incorporated and the identity of the complementary base at the corresponding position of the template sequence bound to the solid support. Therefore, any references herein to the base calling of respective nucleobases at the two portions encompasses the base calling of nucleobases hybridised to the template sequences and, alternatively or additionally, the identification of the corresponding nucleobases of the template sequences. The method may then end at block 1740.

### Selective Processing Methods

The present invention is directed to methods of preparing a polynucleotide strand or strands for identification such that where the strand comprises two portions (in other words, a concatenated polynucleotide sequence comprising a first portion and a second portion) to be identified, or where separate strands each comprise a portion to be identified (in other words, a first polynucleotide sequence comprising a first portion and a second polynucleotide sequence comprising a second portion), such portions can be identified concurrently. This may be achieved by altering the ratio of the different portions which are capable of emitting a signal, which in turn means that during sequencing the signal from the first portion will be greater than the signal from the second portion. It is this difference in the intensity of the first and second signals that allows for the two portions, either on the same or different polynucleotide strands, to be identified simultaneously. It is of course desirable to be able to maximise the throughput and decrease the run time of a sequencing reaction. Concurrent sequencing, achieved by the methods of the present invention, enables at least a doubling of the throughput of a sequencing reaction (i.e. increased sequencing efficiency) as well as a decrease in the time taken to sequence a target polynucleotide strand(s).

Accordingly, we describe a method of preparing at least one polynucleotide sequence (or strand, such terms may be used interchangeably herein) for identification, where the method comprises selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

The at least one polynucleotide sequence comprising the first portion and a second portion may be a plurality of polynucleotide sequences each comprising a first portion and a second portion.

The at least one first polynucleotide sequence comprising a first portion and the at least one second polynucleotide sequence comprising a second portion may be a plurality of first polynucleotide sequences each comprising a first portion, and a plurality of second polynucleotide sequences each comprising a second portion.

Accordingly, the method may comprise selectively processing a plurality of polynucleotide sequences each comprising a first portion and a second portion, or a plurality of first polynucleotide sequences each comprising a first portion and a plurality of second polynucleotide sequences each comprising a second portion, such that a proportion of first portions are capable of generating a first signal and a proportion of second portions are capable of generating a second signal, wherein the selective processing causes an intensity of the first signal to be greater than an intensity of the second signal.

By “identification” is meant here obtaining genetic information from the polynucleotide strand or polynucleotide strands. This may include identification of the genetic sequence of the polynucleotide strand or polynucleotide strands (i.e. sequencing). Furthermore, this may instead, or additionally, include identification of mismatched base pairs. In addition, this may instead, or additionally, include identification of any epigenetic modifications, for example methylation. Accordingly, “identification” may mean identification of the genetic sequence of the polynucleotide strand or polynucleotide strands, mismatched base pairs, and/or identification of any epigenetic modifications.

By “selective processing” is meant here performing an action that changes relative properties of the first portion and the second portion in the at least one polynucleotide sequence comprising a first portion and a second portion (or the plurality of polynucleotide sequences each comprising a first portion and a second portion), or the at least one first polynucleotide sequence comprising a first portion and at least one second polynucleotide sequence comprising a second portion (or the plurality of first polynucleotide sequences each comprising a first portion and the plurality of second polynucleotide sequences each comprising a second portion), so that the intensity of the first signal is greater than the intensity of the second signal. The property may be, for example, a concentration of first portions capable of generating the first signal relative to a concentration of second portions capable of generating the second signal. The action may include, for example, conducting selective amplification, conducting selective sequencing, or preparing for selective sequencing.

The present invention may be applied to a single (concatenated) polynucleotide strand that comprises, on the same strand, a first portion and a second portion to be identified. As explained above, such a strand can be produced using known techniques in the art, such as PCR stitching, tandem insert methods or loop fork methods.

The first portions and second portions may be different polynucleotide sequences. That is, the sequences may be genetically unrelated and/or derived from different sources.

Alternatively, the first portions and second portions may be genetically related.

For example, the first portion may comprise (or be) the forward strand of a polynucleotide sequence (e.g. forward strand of a template), and the second portion may comprise (or be) the reverse strand of the polynucleotide sequence (e.g. reverse strand of the template) or the forward complement strand of the polynucleotide sequence (e.g. forward complement strand of the template). As a further alternative, the first portion may comprise (or be) the reverse strand of a polynucleotide sequence (e.g. reverse strand of a template), and the second portion may comprise (or be) the forward strand of the polynucleotide sequence (e.g. forward strand of the template) or the reverse complement strand of the polynucleotide sequence (e.g. reverse complement strand of the template).

Alternatively, the first portion may comprise (or be) the forward strand of a polynucleotide sequence (e.g. forward strand of a template), and the second portion may comprise (or be) the reverse complement strand of the polynucleotide sequence (e.g. reverse complement strand of the template) (in effect, a reverse complement strand may be considered a “copy” of the forward strand). As a further alternative, the first portion may comprise (or be) the reverse strand of a polynucleotide sequence (e.g. reverse strand of a template), and the second portion may comprise (or be) the forward complement strand of the polynucleotide sequence (e.g. forward complement strand of the template) (in effect, a forward complement may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

The first portion may be referred to herein as read 1 (R1). The second portion may be referred to herein as read 2 (R2).

In embodiments relating to a single (concatenated) polynucleotide strand, the single polynucleotide strand may be attached to a solid support. In one embodiment, this solid support is a flow cell. In one embodiment, the polynucleotide strand is attached to the solid support in a single well of the solid support.

Accordingly, the method may comprise selectively processing at least one polynucleotide sequence comprising a first portion and a second portion, wherein each polynucleotide sequence is attached to a first immobilised primer. In one embodiment, the method may comprise selectively processing a plurality of polynucleotide sequences each comprising a first portion and a second portion, wherein each polynucleotide sequence is attached to a first immobilised primer.

Alternatively, the present invention can be applied to (separate) polynucleotide strands where a first strand comprises a first portion to be identified and a second strand comprises a second portion to be identified.

The first portions and second portions may be different polynucleotide sequences. That is, the sequences may be genetically unrelated and/or derived from different sources.

In one embodiment, the first portion comprises or consists of a sequence derived from a nucleic acid sample (e.g. an insert) and the second portion comprises or consists of a sequence derived from a nucleic acid sample (e.g. an insert).

In one embodiment, the first portion is at least 25 or at least 50 base pairs and the second portion is at least 25 base pairs or at least 50 base pairs.

Alternatively, the first portions and second portions may be genetically related.

For example, the (separate) polynucleotide strands may comprise a first strand that comprises a first portion that may comprise (or be) the forward strand of a polynucleotide sequence (e.g. forward strand of a template), and a second strand that comprises a second portion that may comprise (or be) the reverse strand of the polynucleotide sequence (e.g. reverse strand of the template) or the forward complement strand of the polynucleotide sequence (e.g. forward complement strand of the template). As a further alternative, the (separate) polynucleotide strands may comprise a first strand that comprises a first portion that may comprise (or be) the reverse strand of a polynucleotide sequence (e.g. reverse strand of a template), and a second strand that comprises a second portion that may comprise (or be) the forward strand of the polynucleotide sequence (e.g. forward strand of the template) or the reverse complement strand of the polynucleotide sequence (e.g. reverse complement strand of the template).

Alternatively, the (separate) polynucleotide strands may comprise a first strand that comprises a first portion that may comprise (or be) the forward strand of a polynucleotide sequence (e.g. forward strand of a template), and a second strand that comprises a second portion that may comprise (or be) the reverse complement strand of the polynucleotide sequence (e.g. reverse complement strand of the template) (in effect, a reverse complement strand may be considered a “copy” of the forward strand). As a further alternative, the (separate) polynucleotide strands may comprise a first strand that comprises a first portion that may comprise (or be) the reverse strand of a polynucleotide sequence (e.g. reverse strand of a template), and a second strand that comprises a second portion that may comprise (or be) the forward complement strand of the polynucleotide sequence (e.g. forward complement strand of the template) (in effect, a forward complement strand may be considered a “copy” of the reverse strand). In some embodiments, the first portion may be derived from a forward strand of a target polynucleotide to be sequenced, and the second portion may be derived from a reverse complement strand of the target polynucleotide to be sequenced; or the first portion may be derived from a reverse strand of a target polynucleotide to be sequenced, and the second portion may be derived from a forward complement strand of the target polynucleotide to be sequenced. In these particular embodiments, concurrent sequencing of both the forward and reverse complement strands (or the reverse and forward complement strands) allows mismatched base pairs and/or epigenetic modification to be detected.

Again, the first portion may be referred to herein as read 1 (R1). The second portion may be referred to herein as read 2 (R2).

In embodiments relating to (separate) polynucleotide strands, the first and second strand may be separately attached to a solid support. In one embodiment, this solid support is a flow cell. In one embodiment, each of the first and second strands are attached to the solid support (e.g. flow cell) in a single well of the solid support.

Accordingly, the method may comprise selectively processing at least one first polynucleotide sequence comprising a first portion and at least one second polynucleotide sequence comprising a second portion, wherein each first polynucleotide sequence is attached to a first immobilised primer, and each second polynucleotide sequence is attached to a second immobilised primer. In one embodiment, the method may comprise selectively processing a plurality of first polynucleotide sequences each comprising a first portion and a plurality of at least one second polynucleotide sequences each comprising a second portion, wherein each first polynucleotide sequence is attached to a first immobilised primer, and each second polynucleotide sequence is attached to a second immobilised primer.

The polynucleotide strand or strands may form or be part of a cluster on the solid support.

As used herein, the term “cluster” may refer to a clonal group of template polynucleotides (e.g. DNA or RNA) bound within a single well of a solid support (e.g. flow cell). As such, a cluster may refer to the population of polynucleotide molecules within a well that are then sequenced. A “cluster” may contain a sufficient number of copies of template polynucleotides such that the cluster is able to output a signal (e.g. a light signal) that allows sequencing reads to be performed on the cluster. A “cluster” may comprise, for example, about 500 to about 2000 copies, about 600 to about 1800 copies, about 700 to about 1600 copies, about 800 to 1400 copies, about 900 to 1200 copies, or about 1000 copies of template polynucleotides.

A cluster may be formed by bridge amplification, as described above.

Where the method of the invention involves a single polynucleotide strand with a first and second portion, before sequencing one group of strands (either the group of template polynucleotides, or the group of template complement polynucleotides thereof) may be removed from the solid support, leaving either the templates or the template complements, as explained above. Such a cluster may be considered to be a “monoclonal” cluster.

By “monoclonal” cluster is meant that the population of polynucleotide sequences that are then sequenced (as the next step) are substantially the same—i.e. copies of the same sequence. As such, a “monoclonal” cluster may refer to the population of single polynucleotide molecules within a well that are then sequenced. A “monoclonal” cluster may contain a sufficient number of copies of a single template polynucleotide (or copies of a single template complement polynucleotide) such that the cluster is able to output a signal (e.g. a light signal) that allows sequencing reads to be performed on the “monoclonal” cluster. A “monoclonal” cluster may comprise, for example, about 500 to about 2000 copies, about 600 to about 1800 copies, about 700 to about 1600 copies, about 800 to 1400 copies, about 900 to 1200 copies, or about 1000 copies of a single template polynucleotide (or copies of a single template complement polynucleotide). The copies of the single template polynucleotide (and/or single template complement polynucleotides) may comprise at least about 50%, at least about 60%, at least about 70%, at least about 80%, at least about 90%, or about 95%, 98%, 99% or 100% of all polynucleotides within a single well of the flow cell, and thus providing a substantially monoclonal “cluster”.

Where the method of the invention involves a first polynucleotide strand and a second polynucleotide strand, the cluster formed may be a duoclonal cluster.

By “duoclonal” cluster is meant that the population of polynucleotide sequences that are then sequenced (as the next step) are substantially of two types—e.g. a first sequence and a second sequence. As such, a “duoclonal” cluster may refer to the population of single first sequences and single second sequences within a well that are then sequenced. A “duoclonal” cluster may contain a sufficient number of copies of a single first sequence and copies of a single second sequence such that the cluster is able to output a signal (e.g. a light signal) that allows sequencing reads to be performed on the “monoclonal” cluster. A “duoclonal” cluster may comprise, for example, about 500 to about 2000 combined copies, about 600 to about 1800 combined copies, about 700 to about 1600 combined copies, about 800 to 1400 combined copies, about 900 to 1200 combined copies, or about 1000 combined copies of single first sequences and single second sequences. The copies of single first sequences and single second sequences together may comprise at least about 50%, at least about 60%, at least about 70%, even at least about 80%, at least about 90%, or about 95%, 98%, 99% or 100% of all polynucleotides within a single well of the flow cell, and thus providing a substantially duoclonal “cluster”.

In one embodiment, the selective processing results in the concentration of the first portions capable of generating the first signal being greater than the concentration of the second portions capable of generating the second signal. In other words, the method of the invention results in an altered ratio of R1:R2 molecules, such as within a single cluster or a single well. It is this altered ratio that primes the first portions and second portions to be ready for concurrent sequencing.

The ratio may be between 1.25:1 to 5:1, or between 1.5:1 to 3:1, or about 2:1.

The first signal and the second signal may be spatially unresolved (e.g. generated from the same region or substantially overlapping regions). A first region may be occupied by the at least one first polynucleotide sequence comprising the first portion within the duoclonal cluster is the same as, or substantially overlapping with, a second region occupied by the at least one second polynucleotide sequence comprising the second portion within the duoclonal cluster.

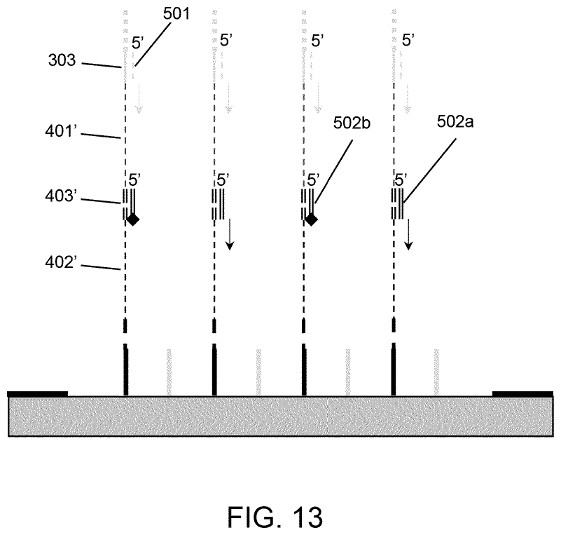

Selective processing may refer to conducting selective sequencing. Alternatively, selective processing may refer to preparing for selective sequencing. As shown in FIG. 13, in one example, selective sequencing may be achieved using a mixture of unblocked and blocked sequencing primers.

Where the method of the invention involves a single (concatenated) polynucleotide strand with a first and second portion, the single (concatenated) polynucleotide strand may comprise a first sequencing primer binding site and a second sequencing primer binding site, where the first sequencing primer binding site and second sequencing primer binding site are of a different sequence to each other and bind different sequencing primers.

Where the method of the invention involves (separate) polynucleotide strands, with a first polynucleotide strand with a first portion, and a second polynucleotide strand with a second portion, the first polynucleotide strand may comprise a first sequencing primer binding site, and the second polynucleotide strand may comprise a second sequencing primer binding site, where the first sequencing primer binding site and second sequencing primer binding site are of a different sequence to each other and bind different sequencing primers.

In one aspect, binding of first sequencing primers to the first sequencing primer site generates a first signal and binding of second sequencing primers to the second sequencing primer site generates a second signal, where the intensity of the first signal is greater than the intensity of the second signal. This may be applied to embodiments where the single (concatenated) polynucleotide strand comprises a first sequencing primer binding site and a second sequencing primer binding site, or to embodiments where the first polynucleotide strand comprises a first sequencing primer binding site, and the second polynucleotide strand comprises a second sequencing primer binding site. In other embodiments, the binding of first sequencing primers and second sequencing primers may not be applied to cases where the first polynucleotide strand comprises a first sequencing primer binding site, and the second polynucleotide strand comprises a second sequencing primer binding site. This is achieved using a mixed population of blocked and unblocked second sequencing primers that bind the second sequencing primer site. Any ratio of blocked:unblocked second primers can be used that generates a second signal that is of a lower intensity than the first signal, for example, the ratio of blocked:unblocked primers may be: 20:80 to 80:20, or 1:2 to 2:1.

For example, a ratio of 50:50 of blocked:unblocked second primers is used, which in turn generates a second signal that is around 50% of the intensity of the first signal.

The first and second sequencing primers may be added to the flow cell at the same time, or separately but sequentially.

By “blocked” is meant that the sequencing primer comprises a blocking group at a 3′ end of the sequencing primer. Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase.

The sequence of the sequencing primers and the sequence primer binding sites are not material to the methods of the invention, as long as the sequencing primers are able to bind to the sequence primer binding site to enable amplification and sequencing of the regions to be identified.

In one embodiment, the first sequencing primer binding site may be selected from ME′-A14′ (as defined in SEQ ID NO: 17 or a variant or fragment thereof), A14′ (as defined in SEQ ID NO: 18 or a variant or fragment thereof), ME′-B15′ (as defined in SEQ ID NO:19 or a variant or fragment thereof) and B15′ (as defined in SEQ ID NO: 20 or a variant or fragment thereof); and the second sequencing primer binding site may be selected from ME′-HYB2 (as defined in SEQ ID NO: 21 or a variant or fragment thereof), HYB2 (as defined in SEQ ID NO: 11 or a variant or fragment thereof), ME′-HYB2′ (as defined in SEQ ID NO: 22 or a variant or fragment thereof) and HYB2′ (as defined in SEQ ID NO: 13 or a variant or fragment thereof).

In another embodiment, the first sequencing primer binding site is ME′-B15′ (as defined in SEQ ID NO: 19 or a variant or fragment thereof), and the second sequencing primer binding site is ME′-HYB2′ (as defined in SEQ ID NO: 22 or a variant or fragment thereof). Alternatively, the first sequencing primer binding site is B15′ (as defined in SEQ ID NO: 20 or a variant or fragment thereof), and the second sequencing primer binding site is HYB2′ (as defined in SEQ ID NO: 13 or a variant or fragment thereof). The first and second sequencing primer sites may be located after (e.g. immediately after) a 3′-end of the first and second portions to be identified.

In another embodiment, the first sequencing primer binding site is ME′-A14′ (as defined in SEQ ID NO: 17 or a variant or fragment thereof), and the second sequencing primer binding site is ME′-HYB2 (as defined in SEQ ID NO: 21 or a variant or fragment thereof). Alternatively, the first sequencing primer binding site may be A14′ (as defined in SEQ ID NO: 18 or a variant or fragment thereof) and the second sequencing primer binding site may be HYB2 (as defined in SEQ ID NO: 11 or a variant or fragment thereof). The first and second sequencing primer sites may be located after (e.g. immediately after) a 3′-end of the first and second portions to be identified.

In one example, the sequencing primer (which may be referred to herein as the second sequencing primer) comprises or consists of a sequence as defined in SEQ ID NO: 11 to 16, or a variant or fragment thereof. The sequencing primer may further comprise a 3′ blocking group as described above to create a blocked sequencing primer. Alternatively, the primer comprises a 3′-OH group. Such a primer is unblocked and can be elongated with a polymerase.

Accordingly, in an aspect of the invention, there is provided a sequencing primer comprising or consisting of a sequence selected from SEQ ID NO: 11 to 16 or a variant or fragment thereof.

In another aspect of the invention there is provided a sequencing composition (also referred to herein as a sequencing mix), comprising a blocked second sequencing primer selected from SEQ ID NO: 15 and 16 or a variant or fragment thereof, and an unblocked second sequencing primer selected from SEQ ID NO: 13 and 14, or a variant or fragment thereof. In one embodiment, the sequencing composition comprises a blocked sequencing primer selected from SEQ ID NO: 15 or a variant or fragment thereof, and an unblocked sequencing primer selected from SEQ ID NO: 13 or a variant or fragment thereof. In another embodiment, the sequencing composition comprises a blocked sequencing primer selected from SEQ ID NO: 16 or a variant or fragment thereof, and an unblocked sequencing primer selected from SEQ ID NO: 14, or a variant or fragment thereof.

In one embodiment, the unblocked and blocked second sequencing primers are present in the sequencing composition in equal concentrations. That is, the ratio of blocked:unblocked second sequencing primers is around 50:50. The sequencing composition may further comprise at least one additional (first) sequencing primer. This additional sequencing primer may be selected from A14-ME (as defined in SEQ ID NO: 9 or a variant or fragment thereof), A14 (as defined in SEQ ID NO: 7 or a variant or fragment thereof), B15-ME (as defined in SEQ ID NO: 10 or a variant or fragment thereof) and B15 (as defined in SEQ ID NO: 8 or a variant or fragment thereof). The sequencing composition may comprise blocked second sequencing primers, unblocked second sequencing primers and at least one first sequencing primer, wherein the first sequencing primer is A14, or B15, or is both A14 and B15.

In another aspect of the invention, there is provided the use of a blocked sequencing primer, in one aspect, a blocked sequencing primer comprising SEQ ID NO: 11 to 16 or a variant or fragment thereof in preparing at least one polynucleotide sequence or a plurality of polynucleotide sequences, for identification.

As shown in FIG. 13, selective sequencing may be conducted on the amplified (monoclonal) cluster shown in FIG. 11F. A plurality of first sequencing primers 501 are added. These first sequencing primers 501 (e.g. B15-ME; or if ME is not present, then B15) anneal to the first terminal sequencing primer binding site 303 (which represents a type of “first sequencing primer binding site”) (e.g. ME′-B15′; or if ME′ is not present, then B15′). A plurality of second unblocked sequencing primers 502a and a plurality of second blocked sequencing primers 502b are added, either at the same time as the first sequencing primers 501, or sequentially (e.g. prior to or after addition of first sequencing primers 501). These second unblocked sequencing primers 502a (e.g. HYB2-ME; or if ME is not present, then HYB2) and second blocked sequencing primers 502b (e.g. blocked HYB2-ME; or if ME is not present, then blocked HYB2) anneal to an internal sequencing primer binding site in the hybridisation sequence 403′ (which represents a type of “second sequencing primer binding site”) (e.g. ME′-HYB2′; or if ME′ is not present, then HYB2′). This then allows the first insert complement sequences 401′ (i.e. “first portions”) to be sequenced and the second insert complement sequences 402′ (i.e. “second portions”) to be sequenced, wherein a greater proportion of first insert complement sequences 401′ are sequenced (grey arrow) compared to a proportion of second insert complement sequences 402′ (black arrow).

Although FIG. 13 shows selective sequencing being conducted on a template strand attached to first immobilised primer 201, in some embodiments the (monoclonal) cluster may instead have template strands attached to second immobilised primer 202. In such a case, the first sequencing primers may instead correspond to A14-ME (or if ME is not present, then A14), and the second unblocked sequencing primers may instead correspond to HYB2′-ME (or if ME is not present, then HYB2′) and second blocked sequencing primers may instead correspond to blocked HYB2′-ME (or if ME is not present, then blocked HYB2′).

In yet other embodiments, the positioning of first sequencing primers and second sequencing primers may be swapped. In other words, the first sequencing binding primers may anneal instead to the internal sequencing primer binding site, and the second sequencing binding primers may anneal instead to the terminal sequencing primer binding site.

FIG. 13 shows concurrent sequencing of a concatenated strand according to the above method. As shown in FIG. 13, a polynucleotide strand with a first portion (insert) and second portion (insert) can be accurately and simultaneously sequenced by a selective sequencing method that uses a mixture of unblocked and blocked sequencing primers as described above.

Alternatively, or in addition, selective processing may refer to selective amplification. That is, selectively amplifying one portion (e.g. the first or second portion) of a single (concatenated) polynucleotide strand or selectively amplifying one portion (e.g. the first or second portion) on a first or second polynucleotide strand.

In one example, selective processing comprises selectively removing some or substantially all of second immobilised primers that have not yet been extended (extended to form a second polynucleotide strand), and conducting at least one further amplification cycle in order to selectively amplify the first polynucleotide sequence(s) relative to the second polynucleotide sequence(s). Immobilised primers that have not yet been extended may be referred to herein as free or un-extended second immobilised primers.

Accordingly, in this example, selective removal of some or substantially all free second immobilised primers is carried out before at least one further round of bridge amplification and before any sequencing of the target regions. As a consequence, the ratio of first polynucleotide capable of generating a first signal to the second polynucleotide that is capable of generating a second signal is altered, which in turn leads to two signals of different intensities, permitting concurrent sequencing of both sequences (or the target regions within those sequences).

By “some or substantially all” is meant that at least 75%, at least 80%, at least 90% or between 95% and 100% of free second immobilised primers are removed.

The selective removal of all or substantially all free second immobilised primers may be carried out using a reagent capable of cleaving the immobilised primer from the solid support. This reagent may be added following at least 5, at least 10, at least 15 or following 20 to 24 rounds of bridge amplification. The reagent may be added separately or together with the amplification reagents for performing the at least one further round of amplification.

As described above, and described in further detail in WO 2008/041002, the first and second immobilised primers may be attached to the surface of a solid support though a linker. The linker may be different for the first and second immobilised primers. The linker may be any cleavable linker; that is the linker may comprise one or more moieties, such as modified nucleotides, that enable selective cleavage of the immobilised primer from the surface of the solid support. By way of non-limiting example, the linker may comprise uracil bases, phosphorothioate groups, ribonucleotides, diol linkages, disulphide linkages, peptides etc. which may be included, not only to allow covalent attachment to a solid support, but also to allow selective cleavage of the linker.

In one example, the first immobilised primer is attached to a solid support though a first linker, where the linker comprises 8-oxoguanine. In this example, free first immobilised primers (that is, primers that are not extended) can be removed using a FPG glycosylase.

In one example, the sequence of the first immobilised primer comprises the following sequence or a variant of fragment thereof:

(SEQ ID NO: 23)
5′-PS-TTTTTTTTTTAATGATACGGCGACCACCGAUCTACAC-3′
where U = 2-deoxyuridine.

In another example, the second immobilised primer is attached to a solid support through a second linker, where the linker comprises uracil or 2-deoxyuridine. In this example, free second immobilised primers (that is, primers that are not extended) can be removed using uracil glycosylase. In one example, free second immobilised primers can be removed using a USER enzyme mix (which is a cocktail of uracil glycosylase and endonuclease VIII). In one example, the sequence of the second immobilised primer comprises the following sequence or a variant of fragment thereof:

(SEQ ID NO: 24)
5′-PS-TTTTTTTTTTCAAGCAGAAGACGGCATACGA[G^{oxo}]AT-3′,
where [G^{oxo}] = 8-oxoguanine.

Accordingly, in a further aspect of the invention, there is provided an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

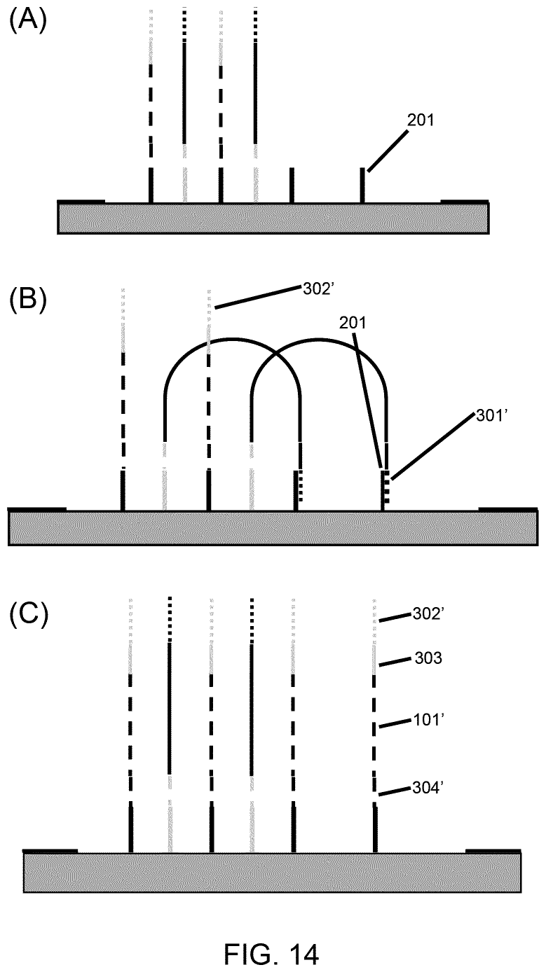

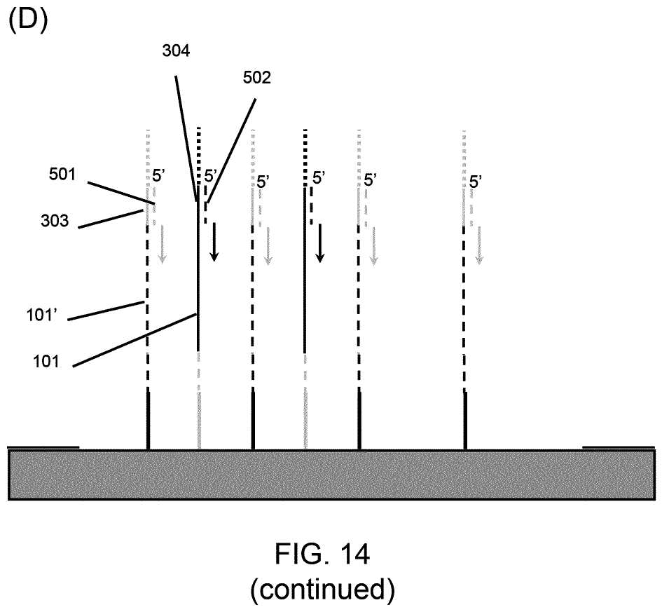

One example of this method is shown in FIG. 14. Selective amplification may be conducted on the amplified (duoclonal) cluster as shown in FIG. 10H. The solid support 200 comprises free first immobilised primers 201 and free second immobilised primers 202. Free second immobilised primers 202 are cleaved from the solid support 200, thus leaving behind free first immobilised primers 201 (FIG. 14A).

The first primer-binding sequence 301′ (e.g. P5′) on one set of template strands may then anneal to the free first immobilised primers 201 (e.g. P5 lawn primer) located within the well 203. By contrast, since free second immobilised primers 202 (e.g. P7 lawn primer) have been removed, second primer-binding sequences 302′ (e.g. P7′) are not able to anneal (FIG. 14B).

After conducting a cycle of bridge amplification, this leads to selective amplification of the template strands comprising the forward strand of the template 101′ and the first terminal sequencing primer binding site 303, relative to the template strands comprising the forward complement strand of the template 101 and the second terminal sequencing primer binding site 304 (FIG. 14C).

Conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 14D).

In another example, selectively processing comprises selectively blocking the extension of some or substantially all of the second immobilised primers that have not yet been extended (extended to form a second polynucleotide strand). Again, these primers may be referred to herein as free or un-extended second immobilised primers. The method may involve using a primer-blocking agent, wherein the primer-blocking agent is configured to limit or prevent synthesis of a strand (i.e. a polynucleotide strand) extending from the second immobilised primer. The method may further involve conducting at least one further amplification cycle. As the free second immobilised primers are blocked from being extended by the primer-blocking agent, only the first immobilised primers can be extended. This leads to amplification of only the first polynucleotide strand (i.e. not the second polynucleotide strand), and as a consequence, an increase in the amount of first polynucleotide sequences relative to the second polynucleotide sequences.

By “some or substantially all” is meant that at least 75%, at least 80%, at least 90%, or between 95% and 100% of free second immobilised primers are blocked.

The primer-blocking agent may be flowed across the solid support following bridge amplification. In one embodiment, the primer-blocking agent is flowed across the solid support following at least 1, 2, 3, 4, 5, 6, 7, 8, 9 or 10 cycles, following at least 15, following at least 20 or following at least 25 rounds of bridge amplification.

In one example, the primer-blocking agent is added whilst first polynucleotide sequence(s) are hybridised to the second immobilised primers. That is, the primer-blocking agent is added during amplification and following extension of at least the first polynucleotide strand. At this stage the extended first polynucleotide strand bends (bridges) and hybridises at its 5′ end to the second immobilised primer. Addition of the primer-blocking agent at this stage prevents extension of the second immobilised primer, which would normally occur using the first polynucleotide strand as its template.

In one embodiment, the primer-blocking agent is a blocked nucleotide. For example, the blocked nucleotide may be A, C, T or G, but may be selected from A or G.

Again, by “blocked” is meant that the sequencing primer comprises a blocking group at a 3′ end of the sequencing primer. Suitable blocking groups include a hairpin loop (e.g. a polynucleotide attached to the 3′-end, comprising in a 5′ to 3′ direction, a cleavable site such as a nucleotide comprising uracil, a loop portion, and a complement portion, wherein the complement portion is substantially complementary to all or a portion of the immobilised primer), a deoxynucleotide, a deoxyribonucleotide, a hydrogen atom instead of a 3′-OH group, a phosphate group, a phosphorothioate group, a propyl spacer (e.g. —O—(CH₂)₃—OH instead of a 3′-OH group)), a modification blocking the 3′-hydroxyl group (e.g. hydroxyl protecting groups, such as silyl ether groups (e.g. trimethylsilyl, triethylsilyl, triisopropylsilyl, t-butyl(dimethyl) silyl, t-butyl(diphenyl) silyl), ether groups (e.g. benzyl, allyl, t-butyl, methoxymethyl (MOM), 2-methoxyethoxymethyl (MEM), tetrahydropyranyl), or acyl groups (e.g. acetyl, benzoyl)), or an inverted nucleobase. However, the blocking group may be any modification that prevents extension (i.e. elongation) of the primer by a polymerase. The block may be reversible or irreversible.

The blocked nucleotide may be added as part of a mixture comprising both blocked and unblocked nucleotides. Alternatively, the blocked nucleotide may be added to the flow cell separately and either before or after unblocked nucleotides are added. Following addition of the blocked nucleotide, at least one more round of bridge amplification is performed.

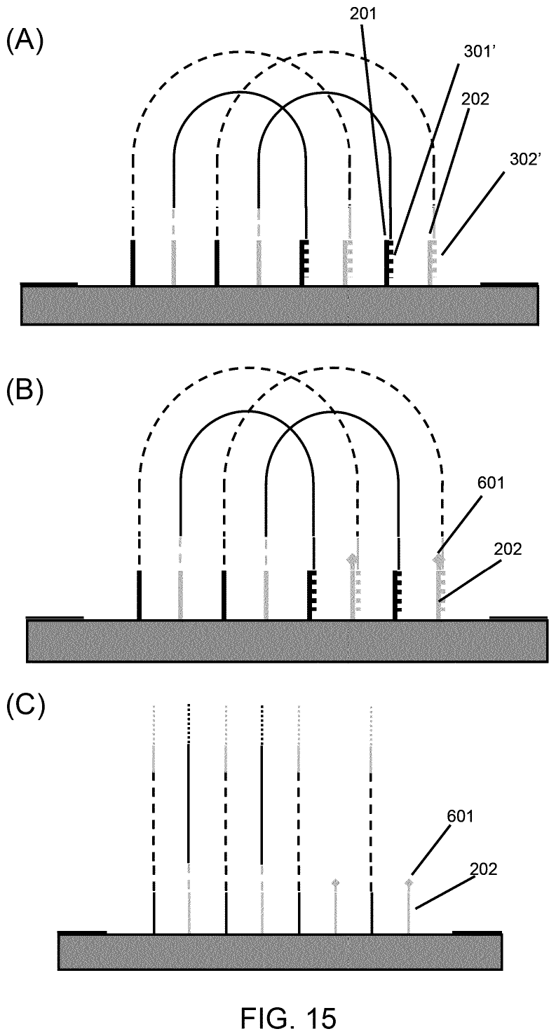

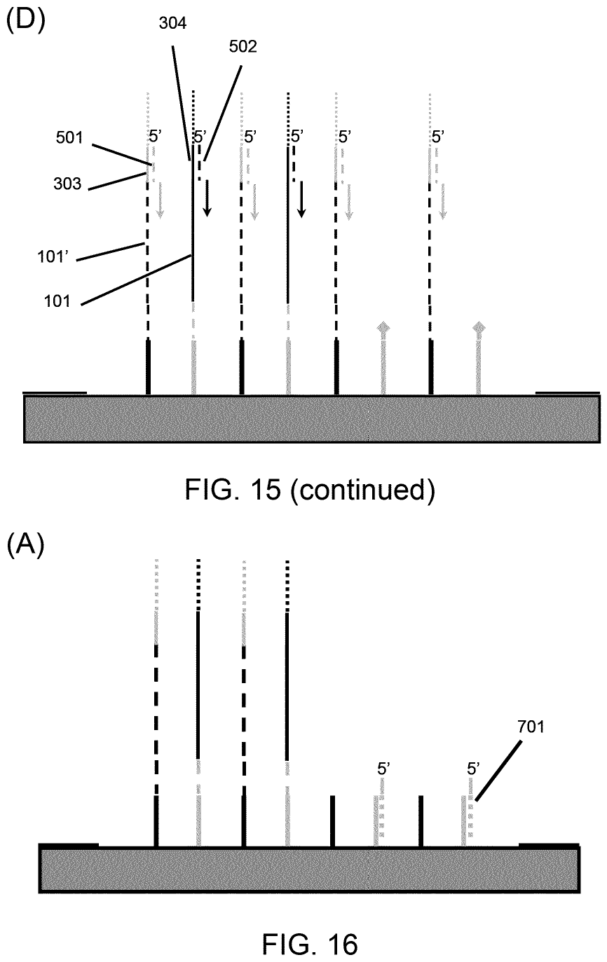

One example of this method is shown in FIG. 15. Selective amplification may be conducted on the amplified (duoclonal) cluster as shown in FIG. 10H. The first primer-binding sequence 301′ (e.g. P5′) on one set of template strands may anneal to first immobilised primers 201 (e.g. P5 lawn primer), and the second primer-binding sequence 302′ (e.g. P7′) on another set of template strands may anneal to second immobilised primers 202 (e.g. P7 lawn primer) (FIG. 15A).

Whilst the second primer-binding sequence 302′ (e.g. P7′) is annealed to the second immobilised primer 202, a primer-blocking agent 601 is selectively installed onto a 3′-end of the second immobilised primer 202, whilst no installation occurs to the 3′-end of the first immobilised primer 201 (FIG. 15B).

Conducting cycle(s) of bridge amplification leads to selective amplification of the template strands comprising the forward strand of the template 101′ and the first terminal sequencing primer binding site 303, relative to the template strands comprising the forward complement strand of the template 101 and the second terminal sequencing primer binding site 304. The primer-blocking agent 601 prevents extension from the second immobilised primer 202. (FIG. 15C).

Conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 15D).

In an alternative example, the method comprises flowing at least one or a plurality of extended primer sequence(s) across the surface of the solid support (e.g. a flow cell), wherein such sequences can bind (e.g. hybridise) free immobilised primers (e.g. P5 or P7) and wherein the extended primer sequences further comprise at least one 5′ additional nucleotide; and (b) adding the primer blocking agent, where the primer blocking agent is complementary to the 5′ additional nucleotide.

The extended primer sequences may be substantially complementary to the first or second immobilised primers (e.g. P5 or P7), or substantially complementary to a portion of the first or second immobilised primer.

The 5′ additional nucleotide may be selected from A, T, C or G, but may be selected from T (or U) or C. In one embodiment, the 5′ additional nucleotide is not a complement of the 3′ nucleotide of the second immobilised primer (where the extended primer sequence binds the first immobilised primer) or is not a complement of the 3′ nucleotide of the first immobilised primer (where the extended primer sequence binds the second immobilised primer). For example, where the first immobilised primer is P5 (for example as defined in SEQ ID NO: 1 or 5) and the second immobilised primer is P7 for example as defined in SEQ ID NO: 2), and where the extended primer sequence binds the first immobilised primer, the 5′ additional nucleotide is not A. Similarly, where the extended primer sequence binds the second immobilised primer, the 5′ additional nucleotide is not G.

In one embodiment, the primer-blocking agent is a blocked nucleotide, for example, as described above. For example, the blocked nucleotide may be A, C, T or G, but may be selected from A or G. Accordingly, where the 5′ additional nucleotide is T or U, the primer-blocking agent is A, and where the 5′ additional nucleotide is C, the primer-blocking agent is G.

Again, the extended primer sequence(s) and primer-blocking agent may be flowed across the solid support following bridge amplification. In one embodiment, the primer-blocking agent may be flowed across the solid support following at least 1, at least 2, at least 3, at least 4, at least 5, at least 6, at least 7, at least 8, at least 9, at least 10, at least 15, at least 20 or at least 25 rounds of bridge amplification.

In one embodiment, the extended primer sequence is selected from SEQ ID NO: 25 to 36 or a variant or fragment thereof.

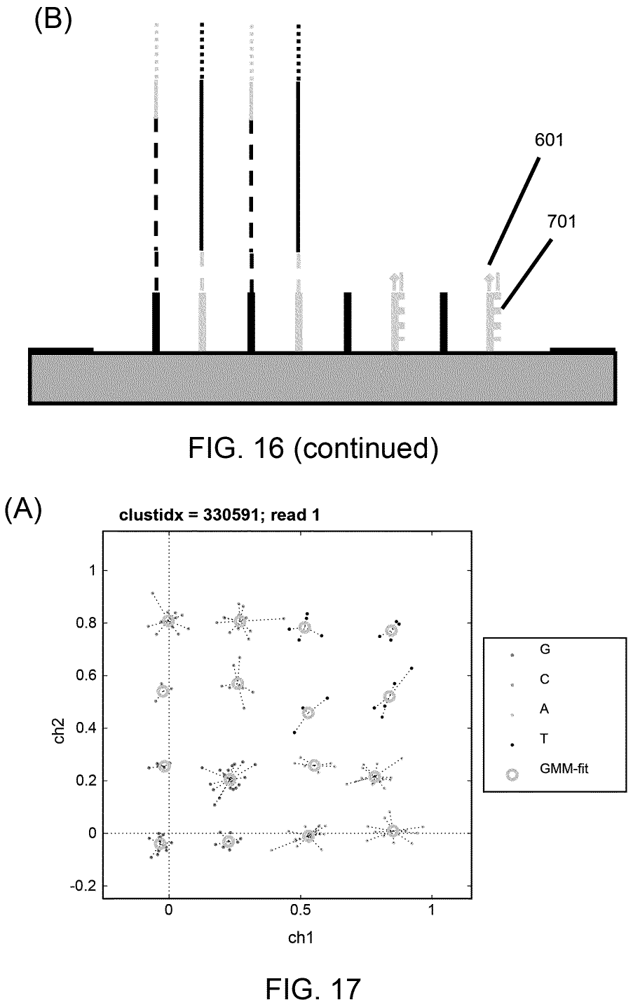

One example of this method is shown in FIG. 16. Selective amplification may be conducted on the amplified (duoclonal) cluster as shown in FIG. 10H; as such following a number of rounds of amplification, a cluster is formed comprising both extended first (e.g. P5) and second (e.g. P7) immobilised polynucleotide strands. Before the next round of amplification, a (or a plurality of) extended primer sequence(s) is flowed across the surface of the solid support 200. The extended primer sequence 701 is substantially complementary to at least a portion, if not all of the immobilised primer (e.g. either P5 or P7) and binds to the immobilised primer (e.g. P5 or P7) as shown in FIG. 16A. As also shown in FIG. 16A, the extended primer sequence 701 comprises at least one additional 5′ nucleotide.

Following addition of the extended primer sequence 701, a primer blocking agent 601 is added and flowed across the surface of the solid support (e.g. flow cell). As the primer-blocking agent 601 is complementary to the 5′ additional nucleotide of the extended primer sequence 701 the primer-blocking agent 601 binds to the 3′-end of the immobilised strands that are hybridised to the extended primer sequence 701, as shown in FIG. 16B. As a consequence, addition of the primer-blocking agent 601 prevents not only extension of the immobilised strand (e.g. P5 or P7) but renders the immobilised primer (P5 or P7) unavailable for hybridisation and subsequent bridge amplification for other extended strands (e.g. 101′) (see FIG. 16B).

Performing at least one more cycle of bridge amplification, leads to selective amplification of the template strands comprising the forward strand of the template 101′ (in a 2:1 ratio of 101′ to 101). Again, similar to FIG. 10D, conducting standard (non-selective) sequencing then allows the forward strands of the template 101′ (i.e. “first portions”) to be sequenced and the forward complement strands of the template 101 (i.e. “second portions”) to be sequenced, wherein a greater proportion of forward strands of the template 101′ are sequenced (grey arrow) compared to a proportion of forward complement strands of the template 101 (black arrow) (FIG. 10D).

The extended primer sequences may be added as part of the amplification mixture described above. Alternatively, the blocked immobilised primer-binding sequence may be added to the flow cell separately and may be before the amplification mixture is added. Following addition of the blocked immobilised primer-binding sequence, at least one more round of bridge amplification is performed.

Accordingly, in a further aspect of the invention, there is provided an extended primer sequence comprising a sequence selected from SEQ ID NO: 25 to 35 or a variant or fragment thereof.

In a further aspect of the invention, there is provided an amplification composition comprising a recombinase, a DNA polymerase, a single-stranded DNA binding protein (SSB) and at least one blocked immobilised-primer-binding sequence.

By “amplification composition” is meant a composition that is suitable for the amplification of a target nucleic acid template.

In another aspect of the invention, there is provided the use of a blocked immobilised-primer-binding sequence, in one embodiment, the blocked immobilised primer-binding sequence may comprise a sequence selected from SEQ ID NO: 25 to 35, in preparing at least one polynucleotide sequence for identification.

### Methods of Sequencing

Also described herein is a method of sequencing at least one polynucleotide sequence, comprising preparing at least one polynucleotide sequence for identification using a method as described herein; and concurrently sequencing nucleobases in the first portion and the second portion based on the intensity of the first signal and the intensity of the second signal.

In one embodiment, sequencing is performed by sequencing-by-synthesis or sequencing-by-ligation.

In one embodiment, the method may further comprise a step of conducting paired-end reads.

The data may be analysed using 16 QAM as mentioned herein.

Accordingly, the step of concurrently sequencing nucleobases may comprise:
(a) obtaining first intensity data comprising a combined intensity of a first signal component obtained based upon a respective first nucleobase at the first portion and a second signal component obtained based upon a respective second nucleobase at the second portion, wherein the first and second signal components are obtained simultaneously;
(b) obtaining second intensity data comprising a combined intensity of a third signal component obtained based upon the respective first nucleobase at the first portion and a fourth signal component obtained based upon the respective second nucleobase at the second portion, wherein the third and fourth signal components are obtained simultaneously;
(c) selecting one of a plurality of classifications based on the first and the second intensity data, wherein each classification represents a possible combination of respective first and second nucleobases; and
(d) based on the selected classification, base calling the respective first and second nucleobases.

In one embodiment, selecting the classification based on the first and second intensity data may comprise selecting the classification based on the combined intensity of the first and second signal components and the combined intensity of the third and fourth signal components.

In one embodiment, the plurality of classifications may comprise sixteen classifications, each classification representing one of sixteen unique combinations of first and second nucleobases.

In one embodiment, the first signal component, second signal component, third signal component and fourth signal component may be generated based on light emissions associated with the respective nucleobase.

In one embodiment, the light emissions may be detected by a sensor, wherein the sensor is configured to provide a single output based upon the first and second signals.

In one embodiment, the sensor may comprise a single sensing element.

In one embodiment, the method may further comprise repeating steps (a) to (d) for each of a plurality of base calling cycles.

### Kits

Methods as described herein may be performed by a user physically. In other words, a user may themselves conduct the methods of preparing at least one polynucleotide sequence for identification as described herein, and as such the methods as described herein may not need to be computer-implemented.

In another aspect of the invention, there is provided a kit comprising instructions for preparing at least one polynucleotide sequence or region of a polynucleotide sequence for identification and/or sequencing at least one polynucleotide sequence or region of a polynucleotide sequence according to the methods described herein.

In one embodiment, the kit may further comprise a sequencing primer comprising or consisting of a sequence selected from SEQ ID NO: 11 to 16 or a variant or fragment thereof.

A sequencing composition comprising a sequencing primer selected from SEQ ID NO: 15 or 16 or a variant or fragment thereof, and a sequencing primer selected from SEQ ID NO: 13 or 14 or a variant or fragment thereof.

In another embodiment, the kit may further comprise an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

In another embodiment, the kit may comprise a primer-blocking agent(s), wherein the primer-blocking agent may be a blocked nucleotide, for example, a blocked A or G. The kit may additionally further comprise at least one extended primer sequence(s), wherein the extended primer sequence is selected from SEQ ID NO: 25 to 35, and wherein the extended primer sequence further comprises a 5′ additional nucleotide, wherein the 5′ additional nucleotide is complementary to the primer-blocking agent.

In another embodiment, the kit may further comprise an amplification mixture comprising a recombinase, a DNA polymerase, a single-stranded DNA binding protein (SSB) and primer-blocking agent, wherein the primer-blocking agent may be a blocked nucleotide, for example, a blocked A or G. In a further embodiment, the kit may additionally comprise at least one extended primer sequence(s), wherein the extended primer sequence is selected from SEQ ID NO: 25 to 35, and wherein the extended primer sequence further comprises a 5′ additional nucleotide, wherein the 5′ additional nucleotide is complementary to the primer-blocking agent.

### Computer Programs and Products

In other embodiments, methods as described herein may be performed by a computer. In other words, a computer may contain instructions to conduct the methods of preparing at least one polynucleotide sequence for identification as described herein, and as such the methods as described herein may be computer-implemented.

Accordingly, in another aspect of the invention, there is provided a data processing device comprising means for carrying out the methods as described herein.

The data processing device may be a polynucleotide sequencer.

The data processing device may comprise reagents used for selective processing methods as described herein.

The data processing device may comprise a solid support, for example, a flow cell.

In another aspect of the invention, there is provided a computer program product comprising instructions which, when the program is executed by a processor, cause the processor to carry out the methods as described herein.

In another aspect of the invention, there is provided a computer-readable storage medium comprising instructions which, when executed by a processor, cause the processor to carry out the methods as described herein.

In another aspect of the invention, there is provided a computer-readable data carrier having stored thereon the computer program product as described herein.

In another aspect of the invention, there is provided a data carrier signal carrying the computer program product as described herein.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Computer-executable instructions may be stored in a (transitory or non-transitory) computer readable storage medium (e.g., memory, storage system, etc.) storing code, or computer readable instructions.

### Additional Notes

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The term “comprising” may be considered to encompass “consisting”.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value. The term “partially” is used to indicate that an effect is only in part or to a limited extent.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

The present invention will now be described by way of the following non-limiting examples.

### EXAMPLES

### Example 1: Concurrent Sequencing of a Concatenated Strand (Different Inserts, Human and PhiX)

### 1.1 Oligo Sequences for Stitch PCR Method:

HYB2-ME—SEQ ID NO: 12; HYB2′-ME—SEQ ID NO: 14

ME sequences are underlined. These were to be used with P5-UDI-A14 and P7-UDI-B15 oligos to PCR up different genomic DNA libraries, making the libraries P5-insert-HYB2′ or P7-insert-HYB2. These libraries were then combined using SOE (splicing by overhang extension) PCR to combine them together. In this experiment the following two oligos were used as partners as examples:

Dual-Biotin 6T-P5-nonlin
(SEQ ID NO: 41)
5′Dual-biotin-TTTTTTAATGATACGGCGACCACCGAGATCTACAC
Dual-Biotin 6T-P7-nonlin
(SEQ ID NO: 42)
5′Dual-biotin-TTTTTTCAAGCAGAAGACGGCATACGAGAT

The 5′ dual biotin is nonetheless, irrelevant for this experiment.

### 1.2 Method

1. Illumina DNA Flex libraries containing human or PhiX (bacteriophage) inserts were prepared following the standard Illumina protocol:
https://emea.illumina.com/products/by-type/sequencing-kits/library-prep-kits/nextera-dna-flex.html

2. Two initial PCRs were set up containing:
25 ul 2× Phusion Mastermix (New England Biolabs)
0.25 ul 100 uM dual-biotin 6T-P5-nonlin
0.25 ul 100 uM HYB2-ME.
1 ul Human Flex library (˜10 ng)
23.5 ul H2O

The other PCR used the dual-biotin 6T-P7-nonlin and HYB2′-ME primer pair on the PhiX Flex library.

3. PCRs were cycled:

98 C for 30 s, followed by 10 cycles of 98 C for 10 s, 50 C for 30 s and 72 C for 30 s, then a 5 min extension step at 72 C and then held at 4 C

4. After checking that material had been made in the initial PCRs via gel electrophoresis, “Splice Overlap Extension” (SOE) PCRs were assembled by combining 20 ul of each of the initial PCRs.

5. SOE PCRs were cycled:

98 C for 30 s, followed by 8 cycles of 98 C for 10 s, 50 C for 60 s and 72 C for 60 s, then a 5 min extension step at 72 C and then held at 4 C.

6. SOE PCRs were cleaned up via a 1×SPRI bead clean-up and quantified using the Qubit Broad Range dsDNA assay (Thermofisher), prior to use in sequencing experiments.

1.3 iSeq100 Sequencing Details:

An iSeq100 cartridge was cracked open, and premixed HCX (90 ul ECX1+45 ul of EXC2+90 ul HCXE3—ExAmp mix for iSeq100) added to the HCX Mixing well. The standard HP10 read 1 primer mix was removed from its well, washed with 200 ul water 5× and then replaced with 150 ul of the 16QAM sequencing primer mix.

16QAM sequencing primer mix—addition of equal concentrations of HYB2′-ME and HYB2′-ME-block in the standard sequencing primer mix from Illumina. The standard sequencing primers are at 0.3 uM each within HP10, and we mix the HYB2′-ME (SEQ ID NO: 14) and HYB2′-ME-block (SEQ ID NO: 16) primers into this to give 0.5 uM of each of these primers. The 50:50 ratio of blocked/unblocked primers for HYB2′-ME gives us the “50%” signal required at this primer site during 16QAM sequencing.

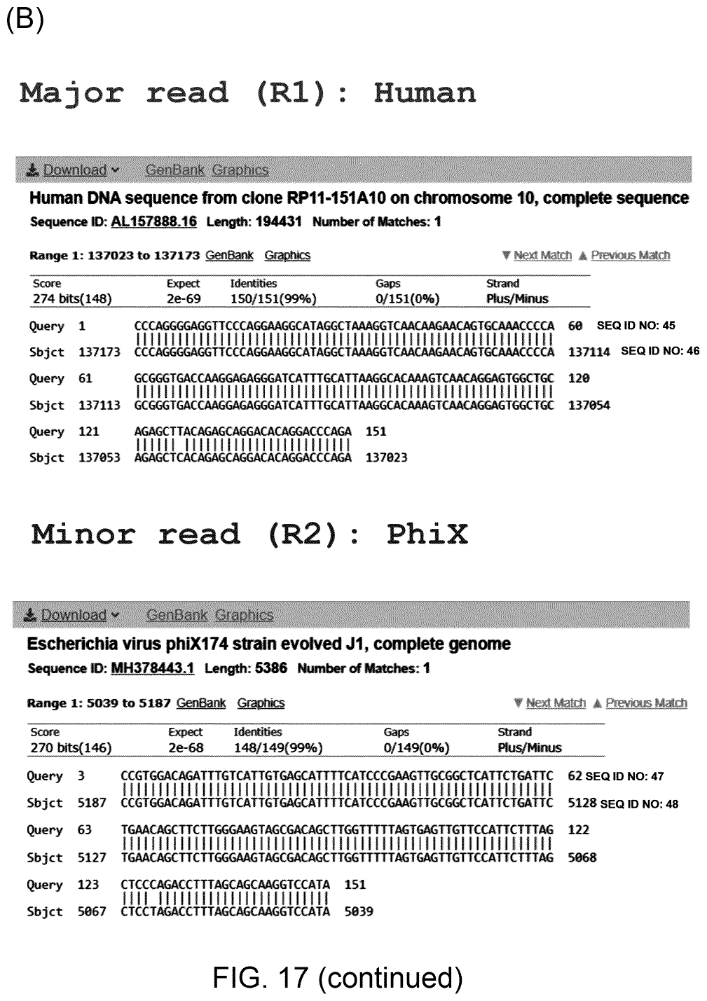

As shown in FIG. 17A, by plotting relative intensities of light signals obtained from a first channel (ch1) and a second channel (ch2), a constellation of 16 clouds is obtained. Each of these clouds allows sequence information to be identified on both the human insert and the PhiX insert, where the top left corner of four clouds corresponds with base calls corresponding to C, the top right corner of four clouds corresponds with base calls corresponding to T, the bottom left corner of four clouds corresponds with base calls corresponding to G, and the bottom right corner of four clouds corresponds with base calls corresponding to A. The basecall read out (R1 and R2) of both the human insert and the PhiX insert is also shown.

As shown in FIG. 17B, alignment of R1 and R2 (minor and major reads respectively) with the known human and PhiX sequence confirmed that the method accurately sequenced the inserts. In particular the sequence identity of R1 and R2 with the known sequences was 99% (150 out of 151 correct base calls for R1 and 148 out of 149 correct base calls for R2).

### Example 2: Concurrent Sequencing of Separate Strands (Forward and Forward Complement, Human)

P5f-Adaptor
(SEQ ID NO: 43)
AATGATACGGCGACCACCGAGATCTACAC*T
P7f-Adaptor
(SEQ ID NO: 44)
CAAGCAGAAGACGGCATACGAGA*T
(* indicates a phosphorothioate linkage)

### 2.1 Method

1. 1 ug mix human genomic DNA from Promega in 50 ul was fragmented to 400-500 bp fragment using the TruSeq-450 program on the Covaris.

2. End prep was performed using NEBNext Ultra II kit.

3. For adapters: 15 ul of F/R oligos from each P5 and P7 were mixed and 1.5 ul 10×NEBuffer 2 was added. The mix was annealed using AK_ANNEAL program (96 C for 2 mins, then to 25 C at −0.1/sec). 30 ul of each of the annealed oligos was then added to 140 ul of water to make 200 ul of 15 uM adapter solution (7.5 uM each side).

4. This mix was used for standard ligation using NEBNext Ultra II kit. The resulting 93 ul was mixed with 3 ul of water and 22.5 ul of iTune (SPRI-like) beads for the 1st size selection cut.

5. Supernatant was then mixed with 12.5 ul of beads for the second size selection. DNA was eluted in 20 ul.

6. 15 ul was used for 6 cycles of Q5 PCR using 10 ul of P5f/P7f primer mix (5 uM each).

7. The PCR product was purified using 0.9× iTune bead selection. It was measured to be at 23 ng/ul, or almost 68 nM.

### 2.2 16QAM Sequencing of Library

The goal is to first block 50% of P7 ends with ddNTP spiked IMX, and then nick P5 end and perform dsSBS sequencing from both ends at the same time (16QAM).

1. ShAdp Human library (with 10% PhiX as a control) was used. After initial denaturation and neutralization of library to give a 20 pM stock, 15 ul of this was added in 485 HT1 to give a 0.6 pM loading concentration for the MiniSeq run.

2. 2.25 ul of 1 mM ddNTPs was added to 500 ul of MiniSeq IMX in the Cust2 position of the MiniSeq cartridge.

3. 250 ul of BMX (Blocking Mix, Illumina) was added to the “EXT” position (cartridge well to the left of the Cust positions).

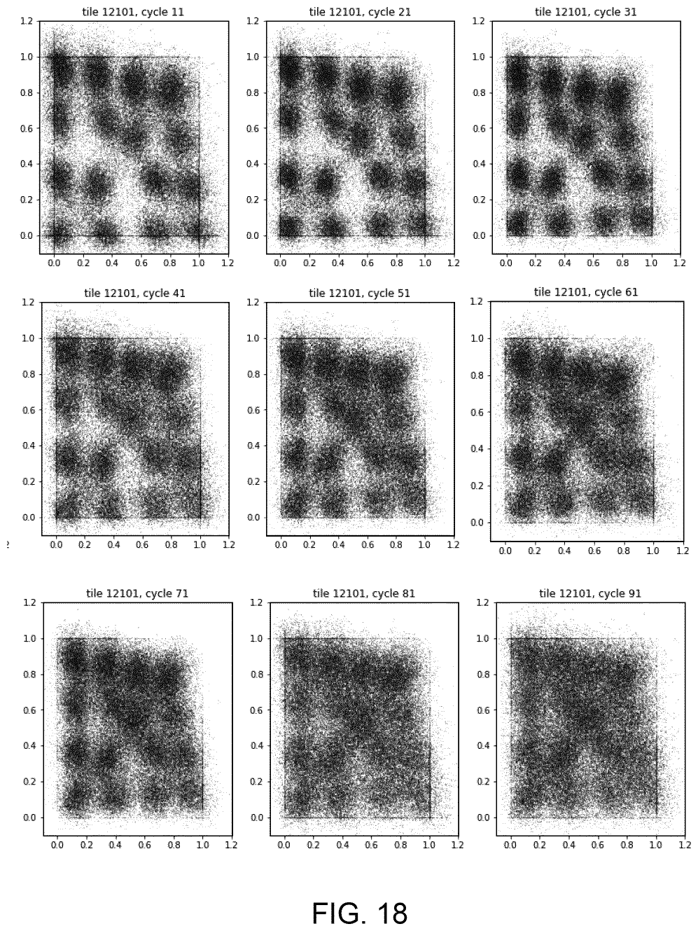

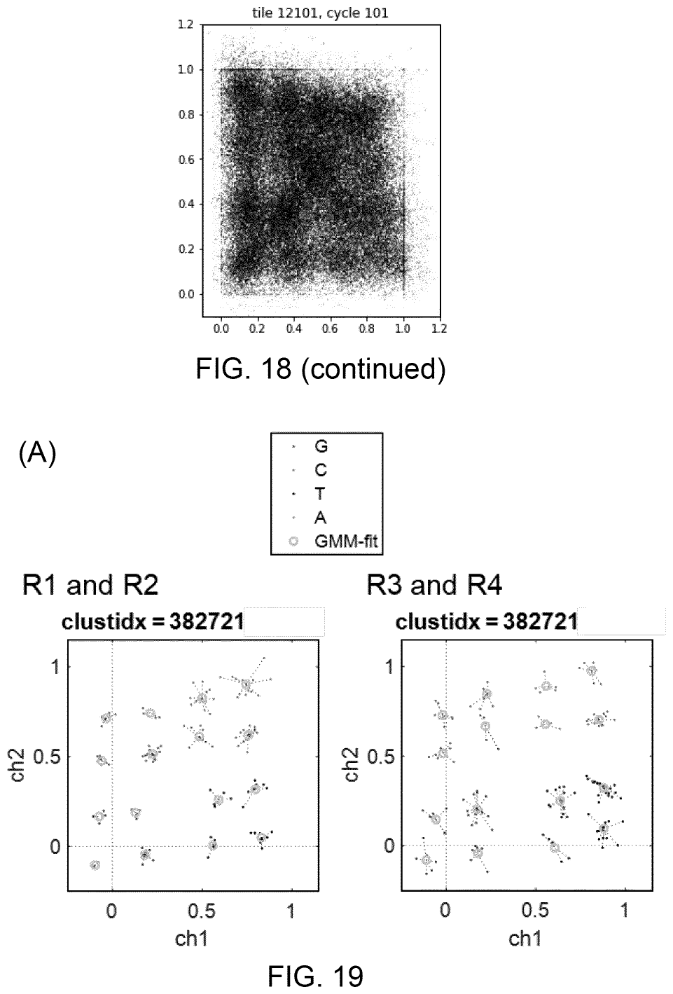

As shown in FIG. 18, by plotting relative intensities of light signals obtained from a first channel (x-axis) and a second channel (y-axis), a constellation of 16 clouds is obtained over multiple cycles. Again, each of these clouds allows sequence information to be identified on both the human insert and the PhiX insert.

### Example 3: Concurrent Sequencing of Separate Strands (Different Strands, Separate Parts of PhiX)

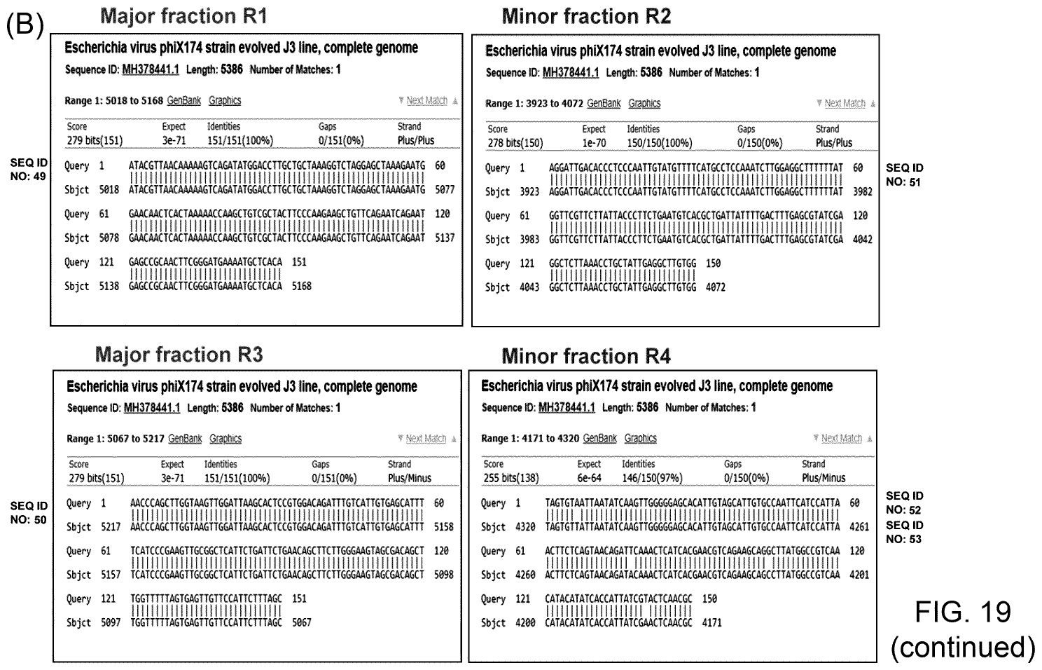

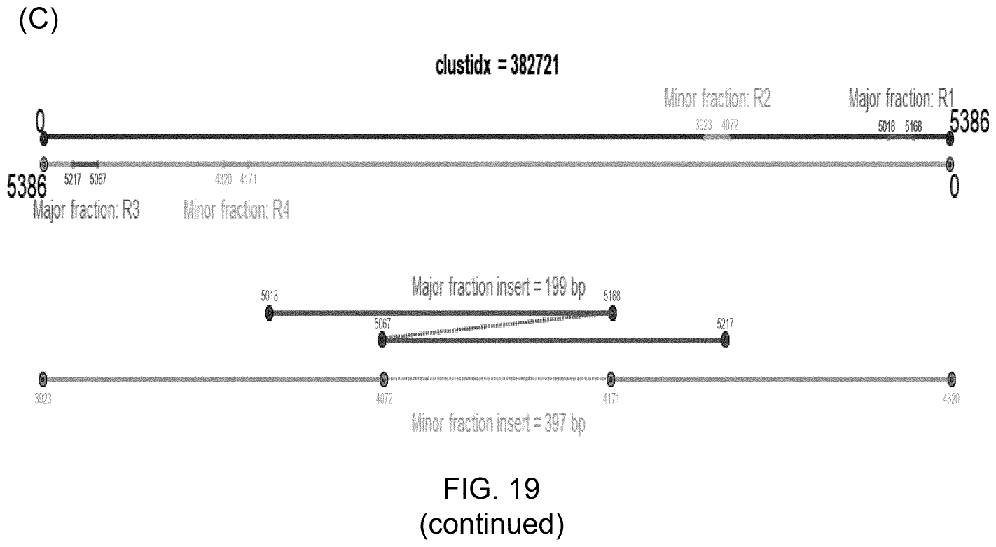

As shown in FIG. 19A, by plotting relative intensities of light signals obtained from a first channel (ch1) and a second channel (ch2), a constellation of 16 clouds is obtained. Each of these clouds allows sequence information to be identified on both the different inserts from the PhiX genome, where the top left corner of four clouds corresponds with base calls corresponding to C, the top right corner of four clouds corresponds with base calls corresponding to A, the bottom left corner of four clouds corresponds with base calls corresponding to G, and the bottom right corner of four clouds corresponds with base calls corresponding to T. The basecall read out (R1 and R2) of both the different inserts from the PhiX genome is also shown.

A subsequent resynthesis step allows “paired end” read to be conducted. This allows a further basecall read out to be obtained (R3 and R4).

As shown in FIG. 19B, alignment of R1, R2, R3 and R4 with the known sequence confirmed that the method accurately sequenced the inserts (in particular the sequence identity of R1, R2 and R3 with the known sequence was 100%).

1. A method of preparing at least one polynucleotide sequence for identification, comprising:
selectively processing at least one polynucleotide sequence comprising a first portion and a second portion, or at least one first polynucleotide sequence comprising a first portion and at least one second polynucleotide sequence comprising a second portion, such that a proportion of first portions are capable of generating a first signal and a proportion of second portions are capable of generating a second signal, wherein the selective processing causes an intensity of the first signal to be greater than an intensity of the second signal.

2. A method according to any one of claim 1, wherein a concentration of the first portions capable of generating the first signal is greater than a concentration of the second portions capable of generating the second signal.

3. A method according to claim 1 or 2, wherein a ratio between the concentration of the first portions capable of generating the first signal and the concentration of the second portions capable of generating the second signal is between 1.25:1 to 5:1, preferably between 1.5:1 to 3:1, more preferably about 2:1.

4. A method according to any preceding claim, comprising selectively processing at least one polynucleotide sequence comprising a first portion and a second portion.

5. A method according to any preceding claim, comprising selectively processing at least one first polynucleotide sequence comprising a first portion and at least one second polynucleotide sequence comprising a second portion.

6. A method according to any preceding claim, wherein the first signal and the second signal are spatially unresolved.

7. A method according to any preceding claim, wherein the at least one polynucleotide sequence comprises portions of a double-stranded nucleic acid template, and each of the first portions comprise a forward strand of the template, and each of the second portions comprise a reverse strand of the template or a forward complement strand of the template; or wherein each of the first portions comprise a reverse strand of the template, and each of the second portions comprise a forward strand of the template or a reverse complement strand of the template.

8. A method according to any preceding claim, wherein the at least one polynucleotide sequence comprises portions of a double-stranded nucleic acid template, and each of the first portions comprises a forward strand of a template, and each of the second portions comprises a reverse complement strand of the template; or wherein each of the first portions comprises a reverse strand of a template, and each of the second portions comprises a forward complement strand of the template.

9. A method according to any preceding claim, wherein the at least one polynucleotide sequence comprising the first portion and the second portion, or the at least one first polynucleotide sequence comprising the first portion and the at least one second polynucleotide sequence comprising the second portion, is/are attached to a solid support, preferably wherein the solid support is a flow cell.

10. A method according to any preceding claim, wherein the at least one polynucleotide sequence comprising the first portion and the second portion, or the at least one first polynucleotide sequence comprising the first portion and the at least one second polynucleotide sequence comprising the second portion, form a cluster on the solid support.

11. A method according to any preceding claim, wherein the cluster is formed by bridge amplification.

12. A method according to any preceding claim, wherein the at least one polynucleotide sequence comprising the first portion and the second portion forms a monoclonal cluster.

13. A method according to any preceding claim, wherein the at least one first polynucleotide sequence comprising the first portion and the at least one second polynucleotide sequence comprising the second portion form a duoclonal cluster.

14. A method according to any preceding claim, wherein a first region occupied by the at least one first polynucleotide sequence comprising the first portion within the duoclonal cluster is the same as, or substantially overlapping with, a second region occupied by the at least one second polynucleotide sequence comprising the second portion within the duoclonal cluster.

15. A method according to any preceding claim, wherein the solid support comprises at least one first immobilised primer and at least one second immobilised primer.

16. A method according to any preceding claim, wherein the first immobilised primer comprises a sequence as defined in SEQ ID NO:1 or 5, or a variant or fragment thereof; and the second immobilised primer comprises a sequence as defined in SEQ ID NO: 2, or a variant or fragment thereof.

17. A method according to any preceding claim, comprising selectively processing at least one polynucleotide sequence comprising a first portion and a second portion, and wherein each polynucleotide sequence is attached to a first immobilised primer.

18. A method according to claim 17, wherein each polynucleotide sequence comprising the first portion and the second portion further comprises a second adaptor sequence, wherein the second adaptor sequence is substantially complementary to the second immobilised primer.

19. A method according to any preceding claim, comprising selectively processing at least one first polynucleotide sequence comprising a first portion and at least one second polynucleotide sequence comprising a second portion, wherein each first polynucleotide sequence is attached to a first immobilised primer, and wherein each second polynucleotide sequence is attached a second immobilised primer.

20. A method according to claim 19, wherein each first polynucleotide sequence comprises a second adaptor sequence and wherein each second polynucleotide sequence comprises a first adaptor sequence, wherein the second adaptor sequence is substantially complementary to the second immobilised primer and wherein the first adaptor sequence is substantially complementary to the first immobilised primer.

21. A method according to any preceding claim, wherein selectively processing comprises preparing for selective sequencing or conducting selective sequencing.

22. A method according to any preceding claim, wherein selectively processing comprises conducting selective amplification.

23. A method according to any preceding claim, wherein selectively processing comprises contacting first sequencing primer binding sites located after a 3′-end of the first portions with first primers and contacting second sequencing primer binding sites located after a 3′-end of the second portions with second primers, wherein the second primers comprises a mixture of blocked second primers and unblocked second primers.

24. A method according to claim 23, wherein the blocked second primer comprises a blocking group at a 3′ end of the blocked second primer.

25. A method according to claim 23 or 24, wherein the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

26. A method according to any one of claims 23 to 25, wherein the blocked second primer comprises a sequence as defined in SEQ ID NO: 11 to 16 or a variant or fragment thereof and/or the unblocked second primer comprises a sequence as defined in SEQ ID NO: 11 to 14 or a variant or fragment thereof.

27. A method according to any one of claims 1 to 22, wherein the selective processing comprises selectively removing some or substantially all of second immobilised primers that are not yet extended, and conducting a further amplification cycle in order to selectively amplify the first polynucleotide sequence(s) relative to the second polynucleotide sequence(s).

28. A method according to any one of claims 1 to 22, wherein selectively processing comprises selectively blocking some or substantially all of second immobilised primers that are not yet extended using a primer blocking agent, wherein the primer blocking agent is configured to limit or prevent synthesis of a strand extending from the second immobilised primer, and conducting a further amplification cycle in order to selectively amplify the first polynucleotide sequence(s) relative to the second polynucleotide sequence(s).

29. A method according to claim 28, wherein the primer blocking agent is added whilst first polynucleotide sequence(s) are hybridised to the second immobilised primers.

30. A method according to claim 28, wherein the method comprises contacting some or substantially all of the second immobilised primers with an extended primer sequence, wherein the extended primer sequence is substantially complementary to the second immobilised primer and further comprises a 5′ additional nucleotide; and adding the primer blocking agent, wherein the primer blocking agent is complementary to the 5′ additional nucleotide.

31. A method according to claim 30, wherein the primer blocking agent is a blocked nucleotide.

32. A method according to claim 31, wherein the blocked nucleotide comprises a blocking group at a 3′ end of the blocked nucleotide.

33. A method according to claim 32, wherein the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

34. A method according to any one of claims 32 to 33, wherein the blocked nucleotide is A or G.

35. A method of sequencing at least one polynucleotide sequence, comprising:
preparing at least one polynucleotide sequence for identification using a method according to any one of claims 1 to 34; and
concurrently sequencing nucleobases in the first portion and the second portion based on the intensity of the first signal and the intensity of the second signal.

36. A method according to claim 35, wherein the step of concurrently sequencing nucleobases comprises performing sequencing-by-synthesis or sequencing-by-ligation.

37. A method according to any one of claims 35 to 36, wherein the method further comprises a step of conducting paired-end reads.

38. A primer, wherein the primer comprises a sequence as defined in SEQ ID NO: 11 to 16, or a variant or fragment thereof.

39. A primer according to claim 38, wherein a 3′ end of the primer comprises a 3′-OH group.

40. A primer according to any one of claims 38 to 39, wherein a 3′ end of the primer comprises a blocking group at a 3′ end of the primer.

41. A primer according to any one of claims 38 to 40, wherein the blocking group is selected from the group consisting of: a hairpin loop, a deoxynucleotide, a deoxyribonucleotide, a hydrogen atom instead of a 3′-OH group, a phosphate group, a phosphorothioate group, a propyl spacer, a modification blocking the 3′-hydroxyl group, or an inverted nucleobase.

42. Use of a primer according to any one of claims 38 to 41 in preparing at least one polynucleotide sequence for identification according to any one of claims 1 to 34.

43. A kit comprising instructions for preparing at least one polynucleotide sequence for identification according to any one of claims 1 to 34; and/or sequencing at least one polynucleotide sequence according to any one of claims 35 to 37.

44. A kit according to claim 43, further comprising a primer according to any one of claims 38 to 41.

45. A kit according to any one of claims 43 to 44, further comprising an amplification mixture comprising a recombinase, a DNA polymerase, a single-stranded DNA binding protein (SSB) and a glycosylase, wherein the glycosylase is either FPG glycosylase or uracil glycosylase or the USER enzyme mix.

46. A kit according to any one of claims 43 to 45, further comprising a primer-blocking agent(s), wherein the primer-blocking agent is preferably a blocked nucleotide, more preferably a blocked A or G.

47. A kit according to any one of claims 43 to 46, further comprising at least one extended primer sequence(s), wherein the extended primer sequence is selected from SEQ ID NO: 25 to 36, and wherein the extended primer sequence further comprises a 5′ additional nucleotide, wherein the 5′ additional nucleotide is complementary to the primer-blocking agent.

48. An amplification composition comprising a recombinase, a DNA polymerase, a single-stranded DNA binding protein (SSB) and primer-blocking agent, wherein the primer-blocking agent is preferably a blocked nucleotide, more preferably a blocked A or G.

49. An amplification composition according to claim 48, wherein the composition further comprises at least one extended primer sequence(s), wherein the extended primer sequence is selected from SEQ ID NO: 25 or 36, and wherein the extended primer sequence comprises a 5′ additional nucleotide, wherein the 5′ additional nucleotide is complementary to the primer-blocking agent.

50. A data processing device comprising means for carrying out a method according to any one of claims 1 to 34 or 35 to 37.

51. A data processing device according to claim 50, wherein the data processing device is a polynucleotide sequencer.

52. A computer program product comprising instructions, which when the program is executed by a processor, cause the processor to carry out a method according to any one of claims 1 to 34 or 35 to 37.

53. A computer-readable storage medium comprising instructions which, when executed by a processor, cause the processor to carry out a method according to any one of claims 1 to 34 or 35 to 37.

54. A computer-readable data carrier having stored thereon a computer program product according to claim 52.

55. A data carrier signal carrying a computer program product according to claim 52.
